SEQUENCE LISTING (6 sequences)

SEQ ID NO: 1 - protein, 452 aa, Homo sapiens
MLACLQRTQNAPGQHLACPSKSLELRKCEAVASAMHSSRYPSPAELDAYAEKVANSPLSIKIFPTNIRVPQHKHLSRTVNGYDTSGQRYSPYPQHTAGYQGLLAIVKAAVSSSSTAAPAGPAKSVLKSAEGKRTKLSPAAVQVGIAPYPVPSTLGPLAYPKPPEAPAPPPGLPAAATAASVIPLPGRGLPLPPSNLPSIHSLLYQLNQQCQAPGAAPPACQGMAIPHPSPAKHGPVPSFPSMAYSAAAGLPDCRKGTELGQGATQALTLAGAAKPAGYADSGLDYLLWPQKPPPPPPQPLRAYSGSTVASKSPEACGGRAYERASGSPLNCGVGLPTSFTVGQYFAAPWNSVLVTPTSDCYNPAAAVVVTELGPGAARELAGPPADALSGLPSKSVCNTSVLSSSLQSLEYLINDIRPPCIKEQMLGKGYETVAVPRLLDHQHAHIRLPVYR

SEQ ID NO: 2 - protein, 80 aa, Artificial Sequence
  Synthetic Construct
MLACLQRTQNAPGQHLACPSKSLELRKCEAVASAMHSSRYPSPAELDAYAEKVANSPLSIKIFPTNIRVPQHKHLSRTVN

SEQ ID NO: 3 - protein, 9 aa, Artificial Sequence
  Synthetic Construct
VANSPLSIK

SEQ ID NO: 4 - protein, 13 aa, Artificial Sequence
  Synthetic Construct
AAVSSSSTAAPAG

SEQ ID NO: 5 - protein, 20 aa, Artificial Sequence
  Synthetic Construct
KIFPTNIRVPQHKHLSRTVN

SEQ ID NO: 6 - protein, 11 aa, Artificial Sequence
  Synthetic Construct
YGRKKRRQRRR

(19) United States
(12) Patent Application Publication
(10) Pub. No.: US 2022/0034913 A1
(43) Pub. Date: Feb. 3, 2022

(54) METHODS AND COMPOSTIONS OF DETECTING AND TREATING NEURODEGENERATIVE DISORDERS

(71) Applicant: CASE WESTERN RESERVE UNIVERSITY, CLEVELAND, OH (US)

(72) Inventor: Xinglong Wang, Cleveland, OH (US)

(21) Appl. No.: 17/435,516

(22) PCT Filed: Mar. 2, 2020
(86) PCT No.: PCT/US2020/020688

§ 371 Date: Sep. 1, 2021

Related U.S. Application Data
(60) Provisional application No. 62/812,601, filed on Mar. 1, 2019.

(51) Int. Cl. G01N 33/68

(52) U.S. Cl. CPC G01N 33/6896; G01N 33/6848; G01N 2800/2835; G01N 2800/2821; G01N 2333/4709

(57) ABSTRACT

A method of identifying a subject at risk of a disease or disorder associated with amyloid aggregation includes assaying for Aggregatin in a bodily sample obtained from the subject, wherein the subject is at risk of having the disease or disorder if the Aggregatin is present above a threshold level.

### RELATED APPLICATION

This application claims priority from U.S. Provisional Application No. 62/812,601, filed Mar. 1, 2019, the subject matter of which is incorporated herein by reference in its entirety.

### GOVERNMENT FUNDING

This invention was made with government support under Grant Nos. RF1AG056320 awarded by The National Institutes of Health and AARG-17-499682, awarded by the U.S. Alzheimer's Association. The United States government has certain rights to the invention.

### BACKGROUND

Alzheimer's disease (AD), the leading cause of dementia, is characterized by pathologic hallmarks amyloid plaques and neurofibrillary tangles, and accompanied by other prominent pathological changes, such as progressive atrophy of the brain, neuropil threads, dystrophic neurites, granulovacuolar degeneration, Hirano bodies, and cerebrovascular amyloid. Amyloid plaques are spherical extracellular lesions composed of amyloid-β (Aβ) peptides, whereas neurofibrillary tangles are intracellular lesions made up of hyperphosphorylated form of the microtubule associated protein tau. Although many risk factors such as aging, lifestyle, and environmental factors are usually considered for the pathogenesis, AD is increasingly proposed to be a genetically dichotomous disease in the early-onset familial form showing classical Mendelian inheritance with little influence from the environment (EOAD), or in the late-onset sporadic form inherited in a non-Mendelian fashion (LOAD).

Less than 10% of AD cases are EOAD with only a small fraction caused by autosomal dominantly inherited genetic changes in amyloid precursor protein (APP), presenilin 1 (PS1) or presenilin 2 (PS2), all of which are responsible for the overproduction of Aβ and the earlier formation of amyloid plaques. Though more than 90% of AD cases are LOAD referred to as sporadic AD without family history, they have the similar clinical and pathologic phenotypes as EOAD and are heritable. In the past decade, intensive efforts have been made to identify over 25 genes associated with AD. In support of the dominant amyloid cascade hypothesis suggesting Aβ deposition in the brain as the primary cause, a number of AD-associated genes are enriched in the APP processing pathway, and involved in Aβ overproduction and amyloid plaque deposition though their encoded proteins are usually not directly associated with amyloid plaques.

### SUMMARY

Embodiments described herein relate to a method of identifying a subject at risk of a disease or disorder associated with amyloid aggregation and/or a method of detecting a disease or disorder associated with amyloid aggregation. The method includes assaying for Aggregatin in a bodily sample obtained from the subject. The subject is at risk of having or has the disease or disorder if the Aggregatin is present in the bodily sample above a threshold level. In other embodiments, the subject is not at risk of having or does not have the disease or disorder if the Aggregatin is not above a threshold level

In some embodiments, the disease or disorder is associated with amyloid β aggregation. The disease or disorder can be a neurodegenerative disease or disorder, such as Alzheimer's disease (AD), Alzheimer's related dementia, Parkinson's disease, Huntington's disease, amyotrophic lateral sclerosis (ALS), Lewy body dementia (LBD), or Down's syndrome.

In other embodiments, the bodily sample can include blood, serum, plasma, urine, cerebrospinal fluid (CSF), synovial fluid, or spinal fluid. The bodily sample can be treated with a protease, such as Lys-C or trypsin, to obtain peptide fragments of Aggregatin, and the presence or level the peptide fragments can be detected by mass-spectrometry to determine the presence or level of Aggregatin in the bodily sample. The peptide fragments can be chromatographically separated from other components in the protease treated sample by liquid chromatography.

In some embodiments, the peptide fragments include peptides having the amino acid sequences of SEQ ID NO: 3 and SEQ ID NO: 4. The ratio of the peptide fragments having SEQ ID NO: 3 and SEQ ID NO: 4 can be determined by mass spectrometry and the determined ratio can be compared with a standard curve generated from mass spectrometric results for known ratios of synthetic peptides having SEQ ID NO: 3 and SEQ ID NO: 4 to determine the presence or level of Aggregatin in the sample.

In other embodiments, the bodily sample is blood, serum, or plasma and the presence of the Aggregatin in the bodily is indicative of the subject being at risk of the disease or disorder.

Other embodiments described herein relate to a method of detecting a disease or disorder associated with amyloid aggregation. The method includes assaying for Aggregatin in a bodily sample obtained from the subject, wherein the subject has the disease or disorder if the Aggregatin is present in the bodily sample above a threshold level. The subject does not have the disease or disorder if the Aggregatin is not above a threshold level.

Still other embodiments relate to a pharmaceutical composition that includes a therapeutic agent. The therapeutic agent includes a synthetic therapeutic peptide of about 10 to about 100 amino acids having an amino acid sequence that is at least about 70%, at least about 75%, at least about 80%, at least about 85%, at least about 90%, or at least about 95% identical to about 10 to about 80 consecutive amino acids of an N-terminal portion of Aggregatin that binds to amyloid (3. The therapeutic peptide includes include an amino acid sequence having SEQ ID NO: 5 and does not induce amyloid β aggregation or promote amyloid deposits.

In some embodiments, the therapeutic peptide includes an amino acid sequence at least about 70%, at least about 75%, at least about 80%, at least about 85%, at least about 90%, or at least about 95% identical to SEQ ID NO: 2. In other embodiments, the therapeutic agent include a transport moiety, such as a TAT peptide, that is directly or indirectly linked to the N-terminal or C-terminal end of the therapeutic peptide.

Other embodiments described herein relate to a method of treating a disease or disorder associated with amyloid aggregation. The method includes administering to the subject a therapeutically effective amount of a therapeutic agent that inhibits Aggregatin induced amyloid β aggregation.

In some embodiments, the disease or disorder is a neurodegenerative disease or disorder. For example, the disease or disorder can include at least one of Alzheimer's disease (AD), frontotemporal dementia, Parkinson's disease, Huntington's disease, amyotrophic lateral sclerosis (ALS), Lewy body dementia (LBD), or Down's syndrome.

In other embodiments, the therapeutic agent includes a synthetic therapeutic peptide of about 10 to about 100 amino acids having an amino acid sequence that is at least about 70%, at least about 75%, at least about 80%, at least about 85%, at least about 90%, or at least about 95% identical to about 10 to about 80 consecutive amino acids of an N-terminal portion of aggregatin that binds to amyloid β. The therapeutic peptide includes an amino acid sequence having SEQ ID NO: 5 and does not induce amyloid β aggregation or promote amyloid deposits.

In some embodiments, the therapeutic peptide includes an amino acid sequence at least about 70%, at least about 75%, at least about 80%, at least about 85%, at least about 90%, or at least about 95% identical to SEQ ID NO: 2. In other embodiments, the therapeutic agent include a transport moiety, such as a TAT peptide, that is directly or indirectly linked to the N-terminal or C-terminal end of the therapeutic peptide.

### BRIEF DESCRIPTION OF THE DRAWINGS

FIGS. 1(A-H) illustrates images and graphs showing expression of Aggregatin in the CNS. (A) Representative immunoblot of Aggregatin by either the Aggregatin antibody (left panel) or Flag antibody (right panel) in HEK293 cells expressing indicated tagged human Aggregatin. The arrow points the faint bands denoting endogenous Aggregatin. (B) Representative immunohistochemistry of GFP in 3 month-old SXFAD mice injected with 2 μl AAV1-shAggregatin or AAV1-shControl into the left and right hippocampal CA1 respectively and scarified 4 weeks later. (C) Representative immunoblot of Aggregatin protein levels in the hippocampus and cerebellum of 3 month-old mice injected with 2 μl AAV1-shControl or AAV1-shAggregatin into the left and right hippocampal CA1 respectively and scarified 4 weeks later. (D and E) Representative immunoblot of Aggregatin protein levels in different tissues of a wild type 6-month old mouse (D) or normal human subject (E). (F-H) Representative immunoblot (F), quantification (F) and statistical analysis (G and H) of Aggregatin levels in AD cortices (n=7 biologically independent samples) compared with age-matched controls (n=7 biologically independent samples). Source data are provided as a Source Data file (Source Data for Statistics and Blots). Data are means±s.e.m. All experiments were independently performed at least three times. For “f”, P-value of logistic regression association analysis between AD and Aggregation expression levels is 0.103 (Odds Ratio=1.61). The analysis includes age and gender as covariates. ns, non-significant. Student's t-test. *P<0.05.

FIGS. 2(A-F) illustrate images showing Aggregatin accumulates within the center of amyloid deposits. (A) Representative images of immunohistochemistry of Aggregatin (arrowheads) and amyloid plaques (stained by the 6E10 antibody) in adjacent sections (denoted by asterisks) of cortices of sporadic AD patients. (B) Representative fluorescent images of Aggregatin, amyloid plaques and DAPI nuclei staining in cortices of sporadic AD. (C) Representative images of immunohistochemistry of Aggregatin (arrowheads) and amyloid plaques (stained by the 6E10 antibody) in adjacent sections (denoted by asterisks) of brains of 6-month-old SXFAD mice. (D) Representative images of Aggregatin, amyloid plaques and DAPI nuclei staining in brains of 6-month-old SXFAD mice. (E, F) Representative dot blots of Aggregatin and Aβ (6E10) in serial fractions of amyloid plaques separated by differential centrifugation in sucrose gradient from sporadic AD patients (E) or 6-month-old SXFAD mice (F). (G, H) Representative immunoblots of Aggregatin and Aβ (6E10) in the SDS-resistant insoluble core-enriched fraction from sporadic AD patients (G) or 6-month old SXFAD mice (H). Arrow heads point Aggregatin. Due to the presence of urea used for plaque core protein extraction, plaque core fractions show slight shifts compared to SDS soluble fraction. All experiments were independently performed at least three times. Source data are provided as a Source Data file (Source Data for Statistics and Blots).

FIGS. 3(A-I) illustrate images showing Aggregatin accumulates within the center of amyloid plaques in AD and APP transgenic mice for AD. (A) Representative images of immunohistochemistry of Aggregatin (arrowheads) and amyloid plaques (stained by the 6E10 antibody) in adjacent sections (denoted by asterisks) of cortices of a familial AD (fAD) patient bearing PS1A246E mutation (fAD_PS1A246E) or a fAD patient bearing APP Swedish mutation (fAD_APPswe). (B and C) Representative images of Aggregatin foci, amyloid plaques (stained by the 6E10 (b) or NU-4 (C) antibody) and DAPI nuclei staining in cortices of sporadic AD (sAD) or fAD patients. (D) Representative images of Aggregatin, amyloid plaques and DAPI nuclei staining in cortices of sporadic AD patients. (E) Representative images of Aggregatin and amyloid plaques in cortices of sporadic AD patients. (F) Representative images of immunohistochemistry of Aggregatin (arrowheads) and amyloid plaques (stained by the 6E10 antibody) in adjacent sections (denoted by asterisks) of brains of 9 month-old APP/PS1, 17 month-old Tg2576 or 17 month 3×Tg mice. (G) Representative images of Aggregatin, amyloid plaques and DAPI nuclei staining in brains of 6 month-old TgCRND8 mice. (H) Representative images of Aggregatin foci, amyloid plaques and DAPI nuclei staining in brains of 6 month-old 5×FAD or TgCRND8 mice. (I) Representative images of immunohistochemistry of Aggregatin (arrowheads) and amyloid plaques (stained by the 6E10 antibody) in 5×FAD mice at different ages.

FIGS. 4(A-N) illustrate the binding of Aggregatin to amyloid plaques or Aβ. (A) Representative images of immunohistochemistry of amyloid plaques (6E10 antibody) and Aggregatin in adjacent 6-month old 5×FAD mouse brain sections pre-incubated with or without 100 nM Flag-tagged rAggregatin or 10 μM Aβ1-42. (B) Representative immunoblot of Aggregatin in brain extracts from control or AD cortices by RIPA buffer. (C) Representative immunoblot (left panel, recognized by the Aggregatin antibody) and Coomassie blue gel staining of 4×Flag-TST tagged rAggregatin. (D) Particle size distribution from dynamic light scattering of rAggregatin. Similar ˜35 nm peaks were observed in both rAggregatin and rAggregatin Δ61-80 groups. (E) Averaged circular dichroism spectra of rAggregatin and rAggregatin Δ61-80 at 1 μM in 10 mM phosphate buffer (pH8.0). Characteristic far-UV CD spectra for an all-α-helix, an all-β-sheet and a random coil protein. The spectrum for an all-α-helix protein has two negative bands of similar magnitude at 222 and 208 nm, and a positive band at ˜190 nm. The spectrum for an all β-sheet protein has in general a negative band between 210-220 nm and a positive band between 195-200 nm. The spectrum for a disorderly (random) protein has a negative band of great magnitude at around 200 nm. CD spectra of Aggregatin showed a negative band of great magnitude at around 200 nm, which is characteristic of an intrinsically disordered protein. Further calculation by K2D3 indicates that the content in α-helix and β-sheet was found to be 2.98% and 33.5% in wild-type Aggregatin, respectively, whereas 2.63% and 33.87% was found in Aggregatin 461-80. (F) Coimmunoprecipitation of purified Flag-tagged rAggregatin and freshly prepared Aβ1-42 without pre-aggregated in vitro (loaded at different amounts). rAggregatin was immunoprecipitated using streptavidin magnetic beads and immunoblotted using the antibody to Flag. (G) Coimmunoprecipitation of purified Flag-tagged rAggregatin and freshly prepared Aβ1-40. rAggregatin was also immunoprecipitated using streptavidin magnetic beads and immunoblotted using the antibody to Flag. (H) Reverse IP experiment. Strep tagged Aβ1-40 or Aβ1-42 was purified by Strevdin-avdin beads from HEK293 cells. rAggregatin (3 μg/reaction) was immunoprecipitated using Aβ1-40 or Aβ1-42 bound streptavidin magnetic beads and immunoblotted using the antibody to Flag. Strep tagged Aβ1-40 or Aβ1-42 was immunoblotted using the 6E10 antibody. (I) Measurement of Aβ1-40 levels bound to immobilized rAggregatin (normalized to maximal rAggregatin and Aβ1-40 binding). n=3 biologically independent samples. (J) Measurement of rAggregatin levels bound to immobilized Aβ1-40 (normalized to maximal rAggregatin and Aβ1-40 binding). n=3 biologically independent samples. (K) Bio-layer interferometry measurement of the binding kinetics of monomeric Aβ1-42 to immobilized BSA (as negative controls). Curves are corresponded to Aβ1-42 at 8320, 4160, 2080, 1040, 520, 260, 130 and 65 nM from the top to bottom. (L) Representative images of immunohistochemistry of amyloid plaques (6E10 antibody) and rAggregatin (Flag antibody) in adjacent 6-month old 5×FAD mouse brain sections pre-incubated with or without 100 nM Flag-tagged rAggregatin and 10 μM Aβ1-42. Asterisks denote landmarks in adjacent sections. (M) Representative images of immunohistochemistry of amyloid plaques (6E10 antibody) and rAggregatin (Flag antibody) in adjacent brain sections of sporadic AD patients pre-incubated with or without 100 nM Flag-tagged rAggregatin and 10 μM Aβ1-42. (N) Representative images of immunohistochemistry of rAggregatin (Flag antibody) in adjacent 6-month old 5×FAD mouse brain sections pre-incubated with or without 100 nM Flag-tagged rAggregatin and 50 μM Aβ1-40. Source data are provided as a Source Data file (Source Data for Statistics and Blots).

FIGS. 5(A-G) illustrate Aggregatin interacts with Aβ. (A) Coimmunoprecipitation of purified Flag-tagged rAggregatin and Aβ1-42 (pre-aggregated in vitro for 24 or 48 h). rAggregatin was immunoprecipitated using streptavidin magnetic beads and immunoblotted using the antibody to Flag. (B) Measurement of Aβ1-42 levels bound to immobilized rAggregatin (normalized to maximal rAggregatin and Aβ1-42 binding). n=3 independent experiments. (C) Measurement of rAggregatin levels bound to immobilized Aβ1-42 (normalized to maximal rAggregatin and Aβ1-42 binding). n=3 independent experiments. (D) Bio-layer interferometry measurement of the binding kinetics of monomeric Aβ1-42 to immobilized rAggregatin. Curves are corresponded to Aβ1-42 at 8320, 4160, 2080, 1040, 520, 260, 130 and 65 nM from the top to bottom. (E, F) Representative immunohistochemistry (E) and quantification (F) of rAggregatin immunoreactivity (Flag antibody) in 5×FAD mouse brain sections after incubation with 100 nM indicated rAggregatin deletion mutants (n=6 independent experiments in each group). Blocks with blue color on the top of each immunohistochemistry image show NABD. (G) Coimmunoprecipitation analysis of purified Flag-tagged rAggregatin deletion mutations and Aβ1-42 using streptavidin magnetic beads. Source data are provided as a Source Data file (Source Data for Statistics and Blots). Data are means±s.e.m (± is the plus-minus sign). One-way analysis of variance (ANOVA) followed by Tukey's multiple comparison test. ****P<0.0001. ns, non-significant.

FIGS. 6(A-D) illustrate the identification of the binding motif of Aggregatin to amyloid plaques. (A and B) Representative immunohistochemistry (A) and quantification (B) of rAggregatin immunoreactivity (Flag antibody) in 5×FAD mouse brain after incubation with 100 nM indicated rAggregatin deletion mutants (n=6 biologically independent samples in each group). (C and D) Representative immunohistochemistry (C) and quantification (D) of rAggregatin immunoreactivity (Flag antibody) in 5×FAD mouse brain co-incubated with 100 nM Flag-tagged rAggregatin and different ratios of Myc-tagged rNABD (i.e., 100 nM, 500 nM and 2,500 nM, n=6 biologically independent samples in each group). Source data are provided as a Source Data file (Source Data for Statistics and Blots). Data are means±s.e.m. One-way analysis of variance (ANOVA) followed by Tukey's multiple comparison test. **P<0.01, ***P<0.001, ****P<0.0001. ns, non-significant.

FIGS. 7(A-H) illustrate Aggregatin accelerates Aβ aggregation in vitro. A ThT-based assay measuring aggregation kinetics of 2.5 μM Aβ1-42 in the presence of various concentrations of rAggregatin (n=5 independent experiments in each time points). B ThT-based assay measuring aggregation kinetics of various concentrations of Aβ1-42 in the presence of 5 nM rAggregatin (n=5 independent experiments in each time points). (C, D) Representative immunoblot and quantification (D) of Aβ1-42 oligomers recognized by 6E10 in the 30 nM rAggregatin and 2.5 μM Aβ1-42 mixture collected after 6-h co-incubation (n=4 independent experiments). Arrow head points to non-specific bands due to long exposure. E, F Representative dot blot (E) and quantification (F) of Aβ1-42 oligomers recognized by the oligomer Aβ specific antibody NU-4 in the 30 nM rAggregatin and 2.5 μM Aβ1-42 mixture collected after 6-hour co-incubation (n=4 independent experiments). (G) Negative staining electron microscopy of 2.5 μM Aβ1-42 aggregates after 0.5-h, 6-h, 2-week, and 4-week co-incubation with or without 30 nM rAggregatin. (H) Representative 3D images of 2.5 μM Aβ1-42 aggregates stained by Thio-S after 4-week co-incubation with or without 30 nM rAggregatin. Source data are provided as a Source Data file (Source Data for Statistics and Blots). Data are means±s.e.m. One-way analysis of variance (ANOVA) followed by Tukey's multiple comparison test. ****P<0.0001. ns, non-significant.

FIGS. 8(A-F) illustrate Aggregatin enhances Aβ aggregation in vitro. (A) ThT-based assay measuring aggregation kinetics of various concentrations of Aβ1-42 indicating that the low concentration of Aβ1-42 at 2.5 μM alone is not sufficient to induce ThT fluorescent increase in vitro (n=5 biologically independent samples in each time points). (B) ThT-based assay measuring aggregation kinetics of the high concentration of Aβ1-42 (10 μM, which alone causes greatly increased ThT fluorescent in vitro, as shown in A) in the presence of various concentrations of rAggregatin (n=5 biologically independent samples in each time points). (C) ThT-based assay measuring aggregation kinetics of Aβ1-40 (15 μM) in the presence of 30 nM rAggregatin (n=5 biologically independent samples in each time points). (D) Representative light exposure of Aβ1-42 oligomers recognized by 6E10 in the 30 nM rAggregatin and 2.5 μM Aβ1-42 mixture collected after 6-hour co-incubation. (E and F) Representative large filed images of negative staining electron microscopy of rAggregatin (no detectable aggregates) or Aβ1-42 (2.5 μM) aggregates 4 weeks after co-incubation with rAggregatin (30 nM). Source data are provided as a Source Data file (Source Data for Statistics and Blots).

FIGS. 9(A-F) illustrate rAggregatin ICV infusion exacerbates amyloid deposits and related neuroinflammation in SXFAD mice. (A) Schematic of rAggregatin ICV infusion. (B) Representative immunoblot of human APP, total APP (human and mouse APP) and BACE1 in brains of 5 month-old mice with ICV infusion of Flag-tagged rAggregatinΔ61-80 or rAggregatin in right half brain at 4 month-old for 4 weeks. (C) Measurements of total Aβ levels in brains of mice with ICV infusion (n=6 biologically independent samples in each group). (D and E) Representative images (D) and quantification (E) of plaque density, load and size by staining a broader range of amyloid plaques using fibrillar dense-core amyloid plaques by Thio-S (D and E) in the total brain (Total), cortex or hippocampus of 5-month old 5×FAD mice with ICV infusion of Flag-tagged rAggregatinΔ61-80 or rAggregatin for 4 weeks (n=18 biologically independent samples in each group). (F) Representative images of astrogliosis (stained by GFAP) and microgliosis (stained by Iba1) in hippocampus of 5-month old 5×FAD mice infused with Flag-tagged rAggregatinΔ61-80 or rAggregatin for 4 weeks. Source data are provided as a Source Data file (Source Data for Statistics and Blots). Data are means±s.e.m. All experiments were independently performed at least three times. Student's t-test or One-way analysis of variance (ANOVA) followed by Tukey's multiple comparison test. ****P<0.0001. ns, non-significant.

FIGS. 10(A-P) illustrate Aggregatin regulates amyloid deposits. 5-month-old 5×FAD mice were ICV infused with Flag-tagged rAggregatinΔ61-80 or rAggregatin for 4 weeks. (A) Representative images of Flag-tagged Aggregatin (Red) and amyloid plaques (Green, Thio-S) in the brain. (B, C) Representative images (B) and quantification (C) of plaque by NU-4 antibody in the total brain (Total), cortex or hippocampus (n=18 mice in each group). (D) Quantification of astrogliosis and microgliosis in hippocampus. (E, F) Y-maze (E) and Barnes maze (f) performance (n=15, 17, 18, 18, and 18 mice for NTG aCSF, NTG rAggregatin, SXFAD aCSF, SXFAD rAggregatinΔ61-80, and SXFAD rAggregatin respectively). 5-month-old SXFAD mice were injected with AAV1-GFP or AAV1-Aggregatin at 1.5 month-old. G, H Representative images (G) and quantification (h) of plaques stained by NU-4 in the hippocampus (n=18 mice in each group). (I) Quantification of astrogliosis and microgliosis in the hippocampus (n=18 mice in each group). (J, K) Y-maze (J) and Barnes maze (K) performance (n=18 mice in each group). 5-month-old SXFAD mice were injected with AAV1-shControl or AAV1-shAggregatin at 1.5-month-old. (L, M) Representative images (H) and quantification (H) of plaques stained by NU-4 in the hippocampus (n=18 mice in each group). (N) Quantification of astrogliosis and microgliosis in the hippocampus (n=18 mice in each group). (O, P) Y-maze (O) and Barnes maze (P) performance (n=18 mice in each group). Source data are provided as a Source Data file (Source Data for Statistics and Blots). Data are means±s.e.m. Student's t-test or one and two-way analysis of variance (ANOVA) followed by Tukey's multiple comparison test. *P<0.05, #P<0.05 (relative to aCSF, AAV1-GFP or shControl AAV1), ***P<0.001, ****P<0.0001. ns, non-significant.

FIGS. 11(A-G) illustrate overexpression of Aggregatin in neurons enhances amyloid deposition and associated neuroinflammation. (A) Schematic diagram of AAV1-Aggregatin and AAV1-GFP. ITR, inverted terminal repeats; eSYN, a hybrid promoter consisting of cytomegalovirus enhancer and human Synapsin I promoter; P2A, porcine teschovirus 2A peptide sequence. P2A autocleavage generates Aggregatin separately from GFP. Right panel shows the representative immunohistochemistry of GFP in 5 month-old 5×FAD mice injected with 2 μl AAV1-Aggregatin into the hippocampal CA1 at 1.5 month-old. (B) Measurements of total Aβ levels in isolated hippocampus of 5 month-old 5×FAD mice injected with 2 μl AAV1-Aggregatin into the hippocampal CA1 at 1.5 month-old. (n=6 biologically independent samples in each group). (C and D) Representative images (C) and quantification (D) of fibrillar dense-core amyloid plaques by Thio-S in the hippocampus of 5 month-old 5×FAD mice injected with AAV1-GFP or AAV1-Aggregatin at 1.5 month-old (n=18 biologically independent samples in each group). (E and F) Quantification of amyloid plaques stained by NU-4 (E) or Thio-S (F) in the brain stem not infected with AAV1 (GFP-negative, n=18 biologically independent samples in each group). (G) Representative images of astrogliosis (stained by GFAP) and microgliosis (stained by Iba1) in the hippocampus of 5 month-old 5×FAD mice injected with AAV1-GFP or AAV1-Aggregatin at 1.5 month-old (n=18 biologically independent samples in each group). Source data are provided as a Source Data file (Source Data for Statistics and Blots). Data are means±s.e.m. Student's t-test or One-way analysis of variance (ANOVA) followed by Tukey's multiple comparison test. ***P<0.001 and ****P<0.0001. ns, non-significant.

FIGS. 12(A-F) illustrates Aggregatin deficiency inhibits amyloid deposition and associated neuroinflammation. (A) Measurements of total Aβ levels in isolated hippocampus of 5 month-old 5×FAD mice injected with 2 μl AAV1-shAggregatin or AAV1-shControl into the hippocampal CA1 at 1.5 month-old. (n=6 biologically independent samples in each group). (B and C) Representative images (B) and quantification (C) of fibrillar dense-core amyloid plaques by Thio-S in the hippocampus of 5 month-old 5×FAD mice injected with AAV1-shAggregatin or AAV1-shControl at 1.5 month-old (n=18 biologically independent samples in each group). (D and E) Quantification of amyloid plaques stained by NU-4 (d) or Thio-S (E) in the brain stem not infected with AAV1 (GFP-negative, n=18 biologically independent samples in each group). (F) Representative images of astrogliosis (stained by GFAP) and microgliosis (stained by Iba1) in the hippocampus of 5 month-old 5×FAD mice injected with AAV1-shControl or AAV1-shAggregatin at 1.5 month-old (n=18 biologically independent samples in each group). Source data are provided as a Source Data file (Source Data for Statistics and Blots). Data are means±s.e.m. Student's t-test or One-way analysis of variance (ANOVA) followed by Tukey's multiple comparison test. ***P<0.001 and ****P<0.0001. ns, non-significant.

FIG. 13 illustrates the presence of Aggregatin in exosomes. Representative Immunoblot of Aggregatin in exosomes isolated from 293 cells expressing Flag tagged Aggregatin. TSG101 was used as the exosome marker. COX IV and Calnexin, markers for mitochondria and ER respectively, were used as negative markers for exosome. Source data are provided as a Source Data file (Source Data for Statistics and Blots).

FIGS. 14(A-C) illustrates ICV of rAggregatin accelerates amyloid deposition in aged 5×FAD mice. (A-C) Representative images (A) and quantification (B and C) of plaque load and size by staining a broader range of amyloid plaques using NU-4 (B) or fibrillar dense-core amyloid plaques by Thio-S(C) in the total brain (Total), cortex or hippocampus of 12-month old 5×FAD mice with ICV infusion of Flag-tagged rAggregatinΔ61-80 or rAggregatin for 4 weeks (n=6 biologically independent samples in each group). Source data are provided as a Source Data file (Source Data for Statistics and Blots). Data are means±s.e.m.

FIGS. 15(A-E) illustrates rNABD (rAggregatin1-80 or rAggregatinΔ81-452) has no effect on Aβ aggregation or amyloid deposits. (A) ThT-based assay measuring aggregation kinetics of Aβ1-42 in the presence of rAggregatin or rNABD indicating that rNABD is not sufficient to induce ThT fluorescent increase in vitro (n=5 biologically independent samples in each time points). (B and C) Representative images (B) and quantification (C) of plaque density, load, and size by staining a broader range of amyloid plaques using NU-4 in the total brain (Total), cortex or hippocampus of 5-month old 5×FAD mice with ICV infusion of Flag-tagged rNABD (i.e., rAggregatinΔ81-452) for 4 weeks (n=18 biologically independent samples in each group). (D and E) Representative images (D) and quantification (E) of the density, load, and size of plaques stained by NU-4 in the hippocampus CA1 of 5 month-old 5×FAD mice injected with AAV1-GFP or AAV1-NABD (i.e., AggregatinΔ81-452) at 1.5 month-old (n=18 biologically independent samples in each group). Source data are provided as a Source Data file (Source Data for Statistics and Blots). Data are means±s.e.m. All experiments were independently performed at least three times. Student's t-test. ns, non-significant.

### DETAILED DESCRIPTION

The embodiments described herein are not limited to the particular methodology, protocols, and reagents, etc., and as such may vary. The terminology used herein is for the purpose of describing particular embodiments only, and is not intended to limit the scope of the present invention, which is defined solely by the claims. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

As used herein, “one or more of a, b, and c” means a, b, c, ab, ac, bc, or abc. The use of “or” herein is the inclusive or.

As used herein, the term “administering” to a patient includes dispensing, delivering or applying an active compound in a pharmaceutical formulation to a subject by any suitable route for delivery of the active compound to the desired location in the subject (e.g., to thereby contact a desired cell such as a desired neuron), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The agents may, for example, be administered to a comatose, anesthetized or paralyzed subject via an intravenous injection or may be administered intravenously to a pregnant subject.

As used herein, the term “amyloid” is intended to denote a protein which is involved in the formation of fibrils, plaques and/or amyloid deposits, either by being part of the fibrils, plaques and/or deposits as such or by being part of the biosynthetic pathway leading to the formation of the fibrils, plaques and/or amyloid deposits.

As used herein, the term “antibody”, includes human and animal mAbs, and preparations of polyclonal antibodies, synthetic antibodies, including recombinant antibodies (antisera), chimeric antibodies, including humanized antibodies, anti-idiotopic antibodies and derivatives thereof. A portion or fragment of an antibody refers to a region of an antibody that retains at least part of its ability (binding specificity and affinity) to bind to a specified epitope. The term “epitope” or “antigenic determinant” refers to a site on an antigen to which antibody binds. Epitopes formed from contiguous amino acids are typically retained on exposure to denaturing solvents whereas epitopes formed by tertiary folding are typically lost on treatment with denaturing solvents. An epitope typically includes at least 3, at least 5, or 8 to 10, or about 13 to 15 amino acids in a unique spatial conformation. Methods of determining spatial conformation of epitopes include, for example, x-ray crystallography and 2-dimensional nuclear magnetic resonance. See, e.g., 66 EPITOPE MAPPING PROTOCOLS IN METS. IN MOLECULAR BIO. (Morris, ed., 1996); Burke et al., 170 J. Inf. Dis. 1110-19 (1994); Tigges et al., 156 J. Immunol. 3901-10).

As used herein, an “effective amount” of an agent or therapeutic peptide is an amount sufficient to achieve a desired therapeutic or pharmacological effect. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

As used herein, the term a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.”

As used herein, the term “gene” or “recombinant gene” refers to a nucleic acid comprising an open reading frame encoding a polypeptide, including both exon and (optionally) intron sequences.

As use herein, the terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

As used herein, the phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the phrases “systemic administration,” “administered systemically,” “peripheral administration” and “administered peripherally” as used herein mean the administration of a compound, drug or other material other than directly into a target tissue (e.g., the central nervous system), such that it enters the animal's system and, thus, is subject to metabolism and other like processes, for example, subcutaneous administration.

As use herein, the term “patient” or “subject” or “animal” or “host” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “polynucleotide sequence” and “nucleotide sequence” are also used interchangeably herein.

As used herein, the terms “peptide” or “polypeptide” are used interchangeably herein and refer to compounds consisting of from about 2 to about 100 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, N.Y., 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

As used herein, the term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of non-natural amino acids).

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

As used herein, the term “recombinant,” as used herein, means that a protein is derived from a prokaryotic or eukaryotic expression system.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of epithelial cells. The term also covers so-called “leaky” promoters, which regulate expression of a selected nucleic acid primarily in one tissue, but cause expression in other tissues as well. The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual (1989); Davis et al., Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical, electrical, and viral-mediated transfection procedures.

As used herein, the terms “transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence), which controls the expression of the recombinant gene in a cell-type in which expression is intended. It will also be understood that the recombinant gene can be under the control of transcriptional regulatory sequences which are the same or which are different from those sequences, which control transcription of the naturally occurring form of a protein.

As used herein, the term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

As used herein, the term “wild type” refers to the naturally-occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The agents, compounds, compositions, antibodies, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

As used herein, the terms “portion”, “fragment”, “variant”, “derivative” and “analog”, when referring to a polypeptide include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

Embodiments described herein relate to a method of identifying a subject at risk of a disease or disorder associated with amyloid aggregation, a method of detecting a disease or disorder associated with amyloid aggregation, a method of treating a disease or disorder associated with amyloid aggregation, and/or pharmaceutical compositions for use in treating diseases or disorders associated with amyloid aggregation.

We found that Aggregatin, the protein encoded by the gene FAM222A, behaves as a plaque core protein directly binding amyloid β (Aβ), facilitating Aβ aggregation, and supporting a pathophysiological role in Alzheimer's disease (AD) onset. In people diagnosed with AD or mild cognitive impairment (MCI), a proportion of whom can progress to AD, FAM222A is associated with the module enriched for atrophy in AD-affected brain regions. FAM222A association with hippocampal volume could be validated in the replication ENIGMA cohort, together pointing to a potential mechanism by which FAM222A may affect regional brain atrophy.

Consistent with the genetic association of FAM222A with longitudinal brain Aβ deposition, pathologically accumulated Aggregatin is readily noted in plaques in AD and amyloid deposits in multiple APP transgenic mice, strongly illustrating the pathological function of Aggregatin. Of note, there are remarkable differences in the morphology of Aggregatin puncta and their co-localization with Aβ. Similarly, as plaques in AD patients are more complex structures than amyloid deposits in APP transgenic mice, it could be expected that Aggregatin is also present differentially in amyloid core-enriched fractions from AD patients and 5×FAD mice. A number of explanations may account for the discrepancy regarding the pattern of Aggregatin puncta or presence of Aggregatin in plaques, including but not limited to differences in disease stages, the effects of Aβ clearance and degradation pathways or the length of time spent for plaque deposition. This notion is indeed supported by the observation that while only one or several condensed Aggregatin foci were present in single plaque in AD, amyloid deposits in cortex from patients with Down's syndrome (DS), a complex genetic abnormality developing AD-like pathology, were largely associated with multiple foci.

Aggregatin appears to bind Aβ1-40 and Aβ1-42 with different affinities. On the basis of the facts that Aggregatin puncta appear concurrently with amyloid plaques and does not exist in the predepositing mice, Aggregatin should accumulate in plaques before or concurrent with rather than after the well formation of plaques.

Aggregatin facilitates Aβ aggregation in vitro although it is not clear whether Aggregatin influences the primary or secondary nucleation. Increasing Aggregatin enhances, whereas reduced Aggregatin suppresses amyloid deposition and associated neuroinflammation and cognitive deficits. Of note, in addition to exacerbate Aβ pathology in adult 5×FAD mice, Aggregatin infusion causes further amyloid deposition in aged 5×FAD mice when amyloid deposit size and number largely plateau. Therefore, Aggregatin is likely an unrecognized co- or even limiting factor both necessary and sufficient for Aβ aggregating into the fibrils to form plaques.

Although the bioinformatics analysis of Aggregatin amino acid sequence reveals that Aggregatin does not contain any known conserved functional motifs, our CD characterization of Aggregatin indicated it as at least a partially folded protein containing α-helix, β-sheet, and intrinsically disordered element(s). We found that Aggregatin was exclusively expressed in the central nervous system (CNS). The substantial loss of Aggregatin in hippocampus does not cause neuronal death, suggesting that Aggregatin may not be vital for neuronal survival.

The genetic inhibition of Aggregatin-Aβ interaction or inhibition of Aggregatin-Aβ interaction using a peptide inhibitor was able to suppress Aggregatin-induced Aβ aggregation or amyloid deposits, suggesting that Aggregatin should directly interact with Aβ to regulate its pathology. Of note, although rNABD (i.e. Aggregatin 1-80 or Aggregatin 481-452) alone is able to bind AP, it does not induce Aβ1-42 aggregation or promote amyloid deposits, suggesting that the C-terminal fragment is also required for Aggregatin-induced Aβ aggregation and plaque formation.

Accordingly, in some embodiments described herein, a method of identifying a subject at risk of a disease or disorder associated with amyloid aggregation and/or a method of detecting a disease or disorder associated with amyloid aggregation can include assaying for Aggregatin in a bodily sample obtained from the subject.

Bodily samples can be obtained from a subject suspected of having a disease or disorder associated with amyloid aggregation or suspected of being at risk of developing a disease or disorder associated with amyloid aggregation and assayed or screened for the presence or level of Aggregatin in the bodily sample. The subject is at risk of having or has the disease or disorder if the Aggregatin is present above a threshold level. In other embodiments, the subject is not at risk of having or does not have the disease or disorder if the Aggregatin is not above a threshold level.

The bodily sample can include, for example, urine, blood, serum, plasma lymph, saliva, cerebrospinal fluid (CSF), synovial fluid, bronchoalveolar lavage (BAL), pericardial fluid, spinal fluid, pleural fluid, pleural effusion, mucus, breast milk, amniotic fluid, vaginal fluid, semen, prostatic fluid, ascitic fluid, peritoneal fluid, aqueous humor, vitreous humor, tears, rheum, perspiration, and cystic fluid. In some embodiments, the bodily sample can include blood, serum, plasma, urine, cerebrospinal fluid (CSF), synovial fluid, or spinal fluid.

The presence or level of Aggregatin in the bodily sample can be detected through a number of distinct approaches. In some cases the bodily sample can be subjected to an immunoassay, such as a western blot using an antibody specific to Aggregatin. In alternate cases, the bodily sample can be subject to peptide digestion followed by mass-spectrometric analysis so as to identify polypeptide constituents of Aggregatin.

In some embodiments, the method includes comparing the amount of the detected Aggregation to a normal control value, wherein an increase in the amount of the Aggregation compared to a normal control value indicates that said patient is suffering from or is at risk of developing the disease or condition.

In some embodiments, the disease or condition is a disease or condition characterized by amyloid aggregation or misfolding. In some embodiments, the disease or condition is an amyloid based disease or condition. In some embodiments, the amyloid-based disease or condition is any disease or condition associated with the increased deposition of amyloid β or amyloid like proteins, such as the presence of amyloid plaques. In some embodiments, the disease is a neuronal disease, for example, a neurodegenerative diseases, in which amyloid β peptides, oligomers, fibrils, or plaques are implicated. For example, Alzheimer's disease, Parkinson's disease, Huntington's disease, amyotrophic lateral sclerosis (ALS), Lewy body dementia (LBD), or Down's syndrome.

A more comprehensive list of disorders characterized by amyloid aggregation into amyloid or protein misfolding includes the following: Alzheimer's disease, Amyloid amyloidosis, Amyloid light chain amyloidosis, amyotrophic lateral sclerosis, apolipoprotein A1, myloidosis, bacterial homeostasis, breast tumors, Cerebral Amyloid Angiopathy, Creutzfeld-Jakob disease, Creutzfeldt-Jacob disease, cystic fibrosis, Diabetes mellitus type 2, Down's syndrome, Familial amyloidotic polyneuropathy, fertility, gastric amyloid deposition, Gaucher's disease, haemodialysis-related amyloidosis, Hereditary non-neuropathic systemic amyloidosis, HIV transmission, Huntington's disease, injection-localized amyloidosis, Lewy body dementia (LBD), lymphoma, Lysozomal storage disorders, lysozyme amyloidosis, nephrogenic diabetes insipidus, p53-related cancers, Parkinson's disease, Pre-eclampsia, protein degradation-related diseases, Rheumatoid arthritis, senile systemic amyloidosis, skin tumors, Spongiform encephalitis, systemic AL amyloidosis, tumoral amyloidosis, and Type II diabetes. In some cases, this list remains partial.

In other embodiments, the disease or disorder associated with amyloid aggregation can include the disease or disorder can include a neurodegenerative disease or disorder, such as neurodegenerative disease or disorder associated neuroinflammation. For example, the disease or disorder can include at least one of Alzheimer's disease (AD), dementia (e.g., frontotemporal dementia), Parkinson's disease, Huntington's disease, amyotrophic lateral sclerosis (ALS), Lewy body dementia (LBD), or Down's syndrome.

In some embodiments, the bodily sample obtained from the subject can be optionally subjected to electrophoresis, such as SDS-page, to isolate Aggregatin in the bodily sample, and then subjected to proteolytic digestion with, for example endoproteinase Lys-C or trypsin, in order to obtain fragments of Aggregatin. These fragments include peptides having the amino acid sequences of SEQ ID NO: 3 and SEQ ID NO: 4. Lys-C is a protease that cleaves proteins on the carboxyl terminal side of lysine residues. This enzyme is naturally found in the bacterium Lysobacter enzymogenes and is commonly used in protein sequencing. Trypsin is a serine protease that cleaves polypeptides at the carboxyl terminal side of lysine or arginine, except when either is followed by proline. Upon proteolytic digestion with Lys-C or trypsin, full-length Aggregatin present in the sample is cleaved to produce several peptides of varying length, including peptides having the amino acid sequences of SEQ ID NO: 3 and SEQ ID NO: 4.

The Aggregatin peptides including peptides having the amino acid sequences of SEQ ID NO: 3 and SEQ ID NO: 4 can be chromatographically separated from other components in the proteolytically cleaved Aggregatin in the bodily sample by liquid chromatography (LC). As used herein, LC refers to a process for the separation of one or more molecules or analytes in a sample from other analytes in the sample. LC involves the slowing of one or more analytes of a fluid solution as the fluid uniformly moves through a column of a finely divided substance. The slowing results from the distribution of the components of the mixture between one or more stationery phases and the mobile phase. LC includes, for example, reverse phase liquid chromatography (RPLC) and high pressure liquid chromatography (HPLC).

As used herein, separation does not necessarily to refer to the removal of all materials other than the analyte, i.e., Aggregatin peptides, from a sample matrix. Instead, the terms are used to refer to a procedure that enriches the amount of one or more analytes of interest relative to one or more other components present in the sample matrix. Such enrichment can include complete removal of other materials, but does not necessarily require such complete removal. Separation techniques can be used to decrease the amount of one or more components from a sample that interfere with the detection of the analyte, for example, by mass spectrometry. For example, a proteolytic fragment(s) with a similar mass-to-charge ratio can interfere with analysis. Therefore, separating on both hydrophobicity and mass-to-charge ratio decreases the likelihood of interference.

In some embodiments, the methods can include analyzing the chromatographically separated Aggregatin peptides by mass spectrometry to determine a ratio of Aggregatin peptides having amino acid sequences of SEQ ID NO: 3 and SEQ ID NO: 4 in the bodily sample. The ratio of the peptide fragments having amino acid sequences of SEQ ID NO: 3 and SEQ ID NO: 4 can be determined by mass spectrometry and the determined ratio can be compared with a standard curve generated from mass spectrometric results for known ratios of synthetic peptides having amino acid sequences of SEQ ID NO: 3 and SEQ ID NO: 4 to determine the presence or level of Aggregatin in the sample.

As used herein, mass spectrometry (MS) analysis refers to a technique for the identification and/or quantitation of molecules in a sample. MS includes ionizing the molecules in a sample, forming charged molecules; separating the charged molecules according to their mass-to-charge ratio and detecting the charged molecules.

MS allows for both the qualitative and quantitative detection of molecules in a sample. The molecules may be ionized and detected by any suitable means known to one of skill in the art. Tandem mass spectrometry (MS/MS), wherein multiple rounds of mass spectrometry occur, either simultaneously using more than one mass analyzer or sequentially using a single mass analyzer can be used to identify molecules in a sample. As used throughout, a mass spectrometer is an apparatus that includes a means for ionizing molecules and detecting charged molecules. Optionally, the tandem mass spectrometer is a quadrupole mass spectrometer. By way of example, the tandem mass spectrometer has an atmospheric pressure ionization source, and the analyzing step comprises an ionization method selected from the group consisting of photo ionization, electro spray ionization (ESI), atmospheric pressure chemical ionization (APCI), electron capture ionization, electron ionization, fast atom bombardment/liquid secondary ionization (F AB/LSI), matrix assisted laser desorption ionization (MALDI), field ionization, field desorption, thermospray/plasmaspray ionization, and particle beam ionization. The ionization method may be in positive ion mode or negative ion mode. The analyzing step may also include multiple reaction monitoring or selected ion monitoring (SIM). Optionally, two or more biomolecules are analyzed simultaneously or sequentially. Optionally, the analyzing step uses a quadrupole analyzer, for example, a triple quadrupole mass spectrometer.

In the methods provided herein, the liquid chromatography column can feed directly or indirectly into the mass spectrometer. Two or more LC columns optionally feed into the same mass spectrometer. In other examples, three or more of the LC columns feed into the same mass spectrometer. Optionally, the mass spectrometer is part of a combined LC-MS system. Any suitable mass spectrometer can be used. Further, a mass spectrometer can be used with any suitable ionization method known in the art. These include, but are not limited to, photoionization, electrospray ionization, atmospheric pressure chemical ionization, atmospheric pressure photoionization, and electron capture ionization.

In the methods described herein, the synthetic Aggregatin peptides having amino acid sequences of SEQ ID NO: 3 and SEQ ID NO: 4 used in any of the methods provided herein can be mass altered or not mass altered. The synthetic Aggregatin peptides having amino acid sequences of SEQ ID NO: 3 and SEQ ID NO: 4 can be mass altered by labeling the peptides with a stable isotope, for example, carbon-13 (¹³C), nitrogen-15 (¹⁵N) or deuterium (²H). For example, and not to be limiting, a synthetic Aggregatin peptide can be synthesized with one or multiple ¹³C-, ¹⁵N-, ²H-labeled amino acids in the desired protease digestion product. The peptide resulting from protease digestion is thereby altered by a known mass as compared to the native peptide. This mass altered peptide can then be spiked at a known concentration into an unknown sample. The mass altered peptide will elute at the same liquid chromatography location as the non-mass altered peptide, thus serving as an internal standard that allows absolute quantification of the amount of Aggregatin in a bodily sample. Synthetic Aggregatin peptides can also be synthesized to incorporate a stable isotope in the desired digestion product in order to quantify the amount of Aggregatin in a bodily sample.

As set forth above, the Aggregatin peptide ratio in the bodily sample can be determined by comparing the mass spectrometric results with a standard curve generated from the mass spectrometric results for protease digests of known ratios of a synthetic Aggregatin peptide comprising SEQ ID NO: 3 to a synthetic Aggregatin peptide comprising SEQ ID NO: 4.

The standard curve is generated by preparing a series of standard solutions, wherein members of the series of standard solutions contain different known ratios of the synthetic Aggregatin peptide comprising SEQ ID NO: 3 and the synthetic Aggregatin peptide comprising SEQ ID NO: 4; incubating the standard solutions of step (a) with a protease, such as Lys-C or trypsin, chromatographically separating by liquid chromatography the synthetic Aggregatin peptides from other components in the incubated solutions; and analyzing by mass spectrometry the chromatographically separated synthetic Aggregatin peptides for each standard solution; (e) determining the mass spectrometric peak volume of the synthetic Aggregatin peptides for each standard solution; and (f) generating a standard curve.

One of skill in the art would know how to prepare a series of standard solutions with different known ratios of the synthetic Aggregatin peptide comprising SEQ ID NO: 3 and the synthetic Aggregatin peptide comprising SEQ ID NO: 4.

In the methods provided herein, mass spec peak volume can be calculated by detecting and determining peak shape for a given mass during elution from an LC-MS system. Since the synthetic Aggregatin peptides have known masses, the intensity of the peaks corresponding to these masses can be tracked during the elution period. Numerous software programs are available for detecting and determining the intensity of these peaks, for example, PeakView 2.2 software available from Sciex (Framingham, Mass.). The methods can further comprise verifying the identity of the peaks by reviewing tandem spectroscopy (MS/MS) results to ensure that the fragmentation pattern corresponds to the predicted fragmentation pattern for the Aggregatin peptides.

Optionally, the method can further include affinity extracting Aggregatin from the biological sample using any affinity extraction technique compatible with the present methodology. In one embodiment, for example, the affinity extraction can be antibody affinity extraction using an antibody selective for Aggregatin. In one embodiment, the antibody is a polyclonal antibody specific for Aggregatin. In another example, the antibody is a monoclonal antibody specific for Aggregatin. Another example of an affinity extraction technique includes aptamer affinity binding. Aptamers are known in the art, and can be single stranded DNA or RNA molecules that can bind to pre-selected targets, including peptides such as Aggregatin with high affinity and specificity.

Various techniques can be utilized with affinity capture, including coupling the binding molecule (e.g. antibody or aptamer) to a solid substrate, followed by collecting the substrate or removing the biological sample from the substrate, depending on the nature of the substrate. For example, the binding molecule can be coupled to a substrate such as magnetic beads, after which the magnetic beads can be mixed with the biological fluid. Following affinity binding of the Aggregatin to the binding molecule, the beads can be collected and washed to remove the biological sample components therefrom.

In some embodiments, a method of detecting Aggregatin correlates with the presence or absence of a disease or disorder associated with aberrant amyloid aggregation or deposition in a subject.

In some embodiments, the presence or detected level of Aggregatin in the bodily sample predicts the presence and or absence of aberrant amyloid aggregation or a disease or disorder associated with amyloid aggregation with greater than with greater than 10%, 15%, 20%, 25%, 30%, 35%, 40%, 45%, 50%, 55%, 60%, 65%, 70%, 75%, 80%, 85%, 90%, 95% or 99% sensitivity. In some embodiments, the presence or detected level of Aggregatin predicts the presence and or absence of aberrant amyloid aggregation or a disease or disorder associated with amyloid aggregation with greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sensitivity.

In some embodiments, the method includes comparing the detected amount of the Aggregatin to a normal control value, wherein an increase in the amount of the Aggregatin compared to a normal control value indicates that a patient is suffering from or is at risk of developing the disease or condition.

In some embodiments, the methods described herein can be used in predicting responsiveness of a patient to a treatment, wherein the method includes bringing a sample suspected to contain Aggregatin, detecting Aggregatin in the sample, and correlating the presence or absence of Aggregatin with the presence or absence of a disease or disorder associate with amyloid aggregation. In some embodiments, the method includes comparing the amount of the detectable Aggregatin before and after onset of the treatment, wherein a decrease in the amount of the detectable Aggregatin indicates that the patient is being responsive to the treatment.

In some embodiments, the methods disclosed herein are used in a test for Alzheimer's disease or the potential to develop Alzheimer's in a human by assaying for the presence of Aggregatin in a blood, serum, or plasma sample for the human, whereby presence of Aggregatin above a threshold is indicative of Alzheimer's or the risk of developing Alzheimer's.

Other embodiments described herein are directed to compositions and methods of treating a disease or disorder associated with amyloid aggregation. The method includes administering to the subject a therapeutically effective amount of a therapeutic agent that decreases, inhibits, reduces, and/or suppresses Aggregatin induced amyloid β aggregation. A decrease, inhibition, reduction, and/or suppression of Aggregatin induced amyloid β aggregation can include any measurable, reproducible, and/or substantial reduction in Aggregatin induced amyloid β aggregation or amyloid deposit associated with microgliosis, astrogliosis, and cognitive impairment.

Aggregatin induced amyloid β aggregation can be decreased, inhibited, reduced, and/or suppressed in several ways including, but not limited to: direct inhibition of the Aggregatin-amyloid β (e.g., by using interfering or inhibiting peptides, dominant negative polypeptides; neutralizing antibodies, small molecules or peptidomimetics), inhibition of genes and/or proteins that facilitate one or more of, the localization, activity, and/or function of the Aggregatin (e.g., by decreasing the expression or activity of the genes and/or proteins, such as FAM222A); introduction of genes and/or proteins that negatively regulate one or more of, activity, and/or function of Aggregatin (e.g., by using recombinant gene expression vectors, recombinant viral vectors or recombinant polypeptides); or gene replacement with, for instance, a hypomorphic mutant of the Aggregatin (e.g., by homologous recombination, overexpression using recombinant gene expression or viral vectors, or mutagenesis).

The therapeutic agent that decreases, inhibits, reduces, or suppresses Aggregatin induced amyloid β aggregation can be delivered systemically and/or locally and once delivered inhibit Aggregatin induced amyloid β aggregation, induced neuronal toxicity, diseases associated Aggregatin induced amyloid β aggregation, and/or aberrant amyloid deposition.

In some embodiments, the therapeutic agent that decreases, inhibits, reduces, or suppresses Aggregatin induced amyloid β aggregation of a subject includes a synthetic therapeutic peptide of about 20 to about 100 amino acids having an amino acid sequence that is at least about 70%, at least about 75%, at least about 80%, at least about 85%, at least about 90%, or at least about 95% identical to about 20 to about 80 consecutive amino acids of an N-terminal portion of Aggregatin that binds to amyloid β. The therapeutic peptide includes an amino acid sequence having SEQ ID NO: 5, can bind to amyloid β, and does not induce amyloid β aggregation or promote amyloid deposits.

In some embodiments, the therapeutic peptide includes an amino acid sequence at least about 70%, at least about 75%, at least about 80%, at least about 85%, at least about 90%, or at least about 95% identical to SEQ ID NO: 2.

The therapeutic peptide can be subject to various changes, substitutions, insertions, and deletions where such changes provide for certain advantages in its use. In this regard, therapeutic peptides that bind to and/or complex with amyloid β and does not induce amyloid β aggregation or promote amyloid deposits can be substantially homologous with, rather than be identical to, the sequence of a recited polypeptide where one or more changes are made and it retains the ability to function.

The therapeutic peptide can be in any of a variety of forms of polypeptide derivatives, that include amides, conjugates with proteins, cyclized polypeptides, polymerized polypeptides, retro-inverso peptides, analogs, fragments, chemically modified polypeptides, and the like derivatives.

Retro-inverso peptides are linear peptides whose amino acid sequence is reversed and the α-center chirality of the amino acid subunits is inverted as well. These types of peptides are designed by including D-amino acids in the reverse sequence to help maintain side chain topology similar to that of the original L-amino acid peptide and make them more resistant to proteolytic degradation. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used as pharmaceuticals. Furthermore, the insertion of D-amino acids in selected sequence regions as sequence blocks containing only D-amino acids or in-between L-amino acids allows the design of peptide based drugs that are bioactive and possess increased bioavailability in addition to being resistant to proteolysis. Furthermore, if properly designed, retro-inverso peptides can have binding characteristics similar to L-peptides.

The term “analog” includes any polypeptide having an amino acid residue sequence substantially identical to a sequence specifically shown herein in which one or more residues have been conservatively substituted with a functionally similar residue and that specifically binds to and/or complexes amyloid f3 as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free

The therapeutic peptides can also be modified by natural processes, such as post translational processing, and/or by chemical modification techniques, which are known in the art. Modifications may occur anywhere in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Therapeutic peptides described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues involve an insertion or a substitution of one or more amino acids.

The therapeutic peptides described herein may be prepared by methods known to those skilled in the art. The peptides may be prepared using recombinant DNA. For example, one preparation can include cultivating a host cell (bacterial or eukaryotic) under conditions, which provide for the expression of peptides and/or proteins within the cell

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic peptides described herein can include additional residues that may be added at either terminus of a polypeptide for the purpose of providing a “linker” by which the polypeptides can be conveniently linked and/or affixed to other polypeptides, proteins, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker can be a flexible peptide linker that links the therapeutic peptide to other polypeptides, proteins, and/or molecules, such as detectable labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

In some embodiments, a therapeutic agent comprising the therapeutic peptides described herein can be provided in the form of a conjugate protein or drug delivery construct includes at least a cell transport subdomain(s) or moiety(ies) (i.e., transport moieties), which is linked to the therapeutic peptide. The transport moieties can facilitate transport of the therapeutic polypeptides into a mammalian (i.e., human or animal) tissue across the blood brain barrier. The transport moieties can be covalently linked to the therapeutic polypeptides. The covalent link can include a peptide bond or a labile bond (e.g., a bond readily cleavable or subject to chemical change in the interior target cell environment). Additionally, the transport moieties can be cross-linked (e.g., chemically cross-linked, UV cross-linked) to the therapeutic polypeptide. The transport moieties can also be linked to the therapeutic polypeptide with linking polypeptides described herein.

The transport moieties can be repeated more than once in the therapeutic agent. The repetition of a transport moiety may affect (e.g., increase) the transport of the peptides and/or proteins by across the blood brain barrier. The transport moiety may also be located either at the amino-terminal region of a therapeutic peptide or at its carboxy-terminal region or at both regions.

In one embodiment, the transport moiety can include at least one transport peptide sequence that allows the therapeutic peptide once linked to the transport moiety to more readily cross the blood brain barrier upon systemic (e.g., intravenous administration). In one example, the transport peptide is a synthetic peptide that contains a Tat-mediated protein delivery sequence (e.g., YGRKKRRQRRR (SEQ ID NO: 6)). The transport peptide can be fused to at least one therapeutic peptides described having a sequence described herein.

Other examples of known transport moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No. 2,301,157. Similarly, HIV Tat protein was shown to be able to cross cellular membranes.

In addition, the transport moiety(ies) can include polypeptides having a basic amino acid rich region covalently linked to an active agent moiety (e.g., intracellular domain-containing fragments inhibitor peptide). As used herein, the term “basic amino acid rich region” relates to a region of a protein with a high content of the basic amino acids such as arginine, histidine, asparagine, glutamine, lysine. A “basic amino acid rich region” may have, for example 15% or more of basic amino acid. In some instance, a “basic amino acid rich region” may have less than 15% of basic amino acids and still function as a transport agent region. In other instances, a basic amino acid region will have 30% or more of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of this application can function as a transport agent region.

In one embodiment, the therapeutic peptide described herein can be non-covalently linked to a transduction agent. An example of a non-covalently linked polypeptide transduction agent is the Chariot protein delivery system (See U.S. Pat. No. 6,841,535; J Biol Chem 274(35):24941-24946; and Nature Biotec. 19:1173-1176, all herein incorporated by reference in their entirety).

In another embodiment, an agent that decreases, inhibits, reduces, and/or suppresses Aggregatin induced amyloid β aggregation, can include an agent that reduces or inhibits Aggregatin expression. “Expression”, means the overall flow of information from a FAM222A gene to produce a gene product, Aggregatin.

In another embodiment, the agent can include an RNAi construct that inhibits or reduces expression of Aggregatin. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the application describes other forms of expression vectors that serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, embodiments tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, a modified ribonucleotide can be introduced by in vitro enzymatic or organic synthesis.

Methods of chemically modifying RNA molecules can be adapted for modifying RNAi constructs (see for example, Nucleic Acids Res, 25:776-780; J Mol Recog 7:89-98; Nucleic Acids Res 23:2661-2668; Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount, which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules described herein can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Proc Natl Acad Sci USA, 98:9742-9747; EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Genes Dev, 2002, 16:948-58; Nature, 2002, 418:38-9; RNA, 2002, 8:842-50; and Proc Natl Acad Sci, 2002, 99:6047-52. Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an example of a vector for bi-directional transcription of a transgene to yield both sense and antisense RNA transcripts of the same transgene in a eukaryotic cell. Accordingly, certain embodiments provide a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In some embodiments, a lentiviral vector can be used for the long-term expression of a siRNA, such as a short-hairpin RNA (shRNA), to knockdown expression of Aggregatin in the brain. Although there have been some safety concerns about the use of lentiviral vectors for gene therapy, self-inactivating lentiviral vectors are considered good candidates for gene therapy as they readily transfect mammalian cells.

By way of example, short-hairpin RNA (shRNA) down regulation of the Aggregatin expression can be created using OligoEngene software (OligoEngine, Seattle, Wash.) to identify sequences as targets of siRNA. The oligo sequences can be annealed and ligated into linearized pSUPER RNAi vector (OligoEngine, Seattle, Wash.) and transformed in E. coli strain DH5α cells. After positive clones are selected, plasmid can be transfected into 293T cells by calcium precipitation. The viral supernatant collected containing shRNA can then be used to infect mammalian cells in order to down regulate Aggregatin.

In another embodiment, the therapeutic agent can include antisense oligonucleotides. Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

The binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein (e.g., Aggregatin).

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups, such as peptides (e.g., for targeting host cell receptors), or agents facilitating transport across the cell membrane (see, e.g., Proc Natl Acad Sci 86:6553-6556; Proc Natl Acad Sci 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., BioTechniques 6:958-976) or intercalating agents. (See, e.g., Pharm Res 5:539-549). To this end, the oligonucleotide may be conjugated or coupled to another molecule.

Oligonucleotides described herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (Nucl. Acids Res. 16:3209), methylphosphonate oligonucleotides can be prepared by use of controlled pore glass polymer supports (Proc Natl Acad Sci 85:7448-7451).

The selection of an appropriate oligonucleotide can be performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense oligonucleotide sufficient to suppress translation on endogenous mRNAs in certain instances. Therefore, another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Expression of the sequence encoding the antisense RNA can be by a promoter known in the art to act in mammalian, preferably human cells. Such promoters can be inducible or constitutive. Such promoters include but are not limited to: the SV40 early promoter region (Nature 290:304-310), the promoter contained in the 3′ long terminal repeat of Rous sarcoma virus (Cell 22:787-797), the herpes thymidine kinase promoter (Proc Natl Acad Sci 78:1441-1445), the regulatory sequences of the metallothionein gene (Nature 296:39-42), etc. A type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

In still other embodiments, the therapeutic agent can include can be an antibody, such as a monoclonal antibody, a polyclonal antibody, or a humanized antibody, that specifically or selectively binds to the N-terminal portion (e.g., SEQ ID NO: 2) of Aggregatin (SEQ ID NO: 1) that binds to amyloid β to inhibit binding of Aggregatin to amyloid β anA ggregatin induced amyloid β Aggregatin and deposition. The antibody can include Fv fragments, single chain Fv (scFv) fragments, Fab′ fragments, F(ab′)2 fragments, single domain antibodies, camelized antibodies and other antibody fragments. The antibody can also include multivalent versions of the foregoing antibodies or fragments thereof including monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and receptor molecules, which naturally interact with a desired target molecule.

In some embodiments the antibody or fragment thereof can specifically or selectively bind to an N-terminal portion of Aggregatin having the amino acid sequence of SEQ ID NO: 2. In other embodiment, the antibody or fragment thereof can specifically bind to an amyloid β binding region of Aggregatin having the amino acid sequence of SEQ ID NO: 5.

Preparation of antibodies can be accomplished by any number of methods for generating antibodies. These methods typically include the step of immunization of animals, such as mice or rabbits, with a desired immunogen (e.g., a desired target molecule or fragment thereof). Once the mammals have been immunized, and boosted one or more times with the desired immunogen(s), antibody-producing hybridomas may be prepared and screened according to well known methods. See, for example, Kuby, Janis, Immunology, Third Edition, pp. 131-139, W.H. Freeman & Co. (1997), for a general overview of monoclonal antibody production, that portion of which is incorporated herein by reference.

In vitro methods that combine antibody recognition and phage display techniques can also be used to allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods.

In some embodiments, phage display technology may be used to generate an antibody or fragment thereof specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding a scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as a Fab′ fragment, may be cloned into phage display vectors for surface expression on phage particles. The phage may be any phage, such as lambda, but typically is a filamentous phage, such as Fd and M13, typically M13.

In phage display vectors, the V_{H}-linker-V_{L} sequence is cloned into a phage surface protein (for M13, the surface proteins g3p (pIII) or g8p, most typically g3p). Phage display systems also include phagemid systems, which are based on a phagemid plasmid vector containing the phage surface protein genes (for example, g3p and g8p of M13) and the phage origin of replication. To produce phage particles, cells containing the phagemid are rescued with helper phage providing the remaining proteins needed for the generation of phage. Only the phagemid vector is packaged in the resulting phage particles because replication of the phagemid is grossly favored over replication of the helper phage DNA. Phagemid packaging systems for production of antibodies are commercially available. One example of a commercially available phagemid packaging system that also permits production of soluble ScFv fragments in bacterial cells is the Recombinant Phage Antibody system (RPAS), commercially available from Amersham Pharmacia Biotech, Inc. of Piscataway, N.J. and the pSKAN Phagemid Display System, commercially available from MoBiTec, LLC of Marco Island, Fla. Phage display systems, their construction, and screening methods are described in detail in, among others, U.S. Pat. Nos. 5,702,892, 5,750,373, 5,821,047 and 6,127,132, each of which is incorporated herein by reference in their entirety.

In some embodiments, a therapeutic amount of the therapeutic agent can be administered to a subject to inhibit Aggregatin induced amyloid β aggregation and treat a disease or disorder associated with amyloid aggregation. In some embodiments, the disease or disorder is a neurodegenerative disease or disorder. For example, the disease or disorder can include at least one of Alzheimer's disease (AD), dementias related to Alzheimer's disease, frontotemporal dementia, Parkinson's disease, Huntington's disease, amyotrophic lateral sclerosis (ALS), Lewy body dementia (LBD), or Down's syndrome.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

The therapeutic agents described herein may further be modified (e.g., chemically modified). Such modification may be designed to facilitate manipulation or purification of the molecule, to increase solubility of the molecule, to facilitate administration, targeting to the desired location, to increase or decrease half life. A number of such modifications are known in the art and can be applied by the skilled practitioner.

In another aspect, the therapeutic agents can be provided in pharmaceutical compositions. The pharmaceutical compositions will generally comprise an effective amount of agent, dissolved or dispersed in a pharmaceutically acceptable carrier or aqueous medium. Combined therapeutics are also contemplated, and the same type of underlying pharmaceutical compositions may be employed for both single and combined medicaments.

In some embodiments, the therapeutic agents can be formulated for parenteral administration, e.g., formulated for injection via the subcutaneous, intravenous, intramuscular, transdermal, intravitreal, or other such routes, including peristaltic administration and direct instillation into targeted site. The preparation of an aqueous composition that contains such a therapeutic agent as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms that can be used for injectable use include sterile aqueous solutions or dispersions; formulations including sesame oil, peanut oil or aqueous propylene glycol; and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

Compositions of the therapeutic agents can be formulated into a sterile aqueous composition in a neutral or salt form. Solutions as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Examples of carriers include solvents and dispersion media containing, for example, water, ethanol, polyol (for example, glycerol, propylene glycol, and liquid polyethylene glycol, and the like), suitable mixtures thereof, and vegetable oils. In many cases, it will be preferable to include isotonic agents, for example, sugars or sodium chloride. The proper fluidity can be maintained, for example, by the use of a coating, such as lecithin, by the maintenance of the required particle size in the case of dispersion and/or by the use of surfactants.

Under ordinary conditions of storage and use, all such preparations should contain a preservative to prevent the growth of microorganisms. The prevention of the action of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate and gelatin.

Prior to or upon formulation, the therapeutic agents can be extensively dialyzed to remove undesired small molecular weight molecules, and/or lyophilized for more ready formulation into a desired vehicle, where appropriate. Sterile injectable solutions are prepared by incorporating the active agents in the required amount in the appropriate solvent with various of the other ingredients enumerated above, as desired, followed by filtered sterilization. Generally, dispersions are prepared by incorporating the various sterilized active ingredients into a sterile vehicle that contains the basic dispersion medium and the required other ingredients from those enumerated above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Examples of pharmaceutical compositions can generally include an amount of the therapeutic agent admixed with an acceptable pharmaceutical diluent or excipient, such as a sterile aqueous solution, to give a range of final concentrations, depending on the intended use.

Formulation of the pharmaceutical compounds for use in the modes of administration noted above (and others) are known in the art and are described, for example, in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y., U.S.A., 2005; and Mathiowitz et al., eds., Bioadhesive Drug Delivery Systems, Drugs and the Pharmaceutical Sciences Series, Marcel Dekker, Inc., N.Y., U.S.A., 1999. Compounds of the invention can be formulated into pharmaceutical compositions containing pharmaceutically acceptable non-toxic excipients and carriers. The excipients are all components present in the pharmaceutical formulation other than the active ingredient or ingredients. Suitable excipients and carriers are composed of materials that are considered safe and effective and may be administered to an individual without causing undesirable biological side effects, or unwanted interactions with other medications. Suitable excipients and carriers are those, which are composed of materials that will not affect the bioavailability and performance of the agent. As generally used herein “excipient” includes, but is not limited to surfactants, emulsifiers, emulsion stabilizers, emollients, buffers, solvents, dyes, flavors, binders, fillers, lubricants, and preservatives. Suitable excipients include those generally known in the art such as the “Handbook of Pharmaceutical Excipients”, 4th Ed., Pharmaceutical Press, 2003.

Formulations of the therapeutic agents are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, topical formulations, liposomal forms and the like. The type of form for administration will be matched to the disease or disorder to be treated.

Pharmaceutical “slow release” capsules or “sustained release” compositions or preparations may be used and are generally applicable. Slow release formulations are generally designed to give a constant drug level over an extended period and may be used to deliver a TDP-43 mitochondrial localization inhibitor peptide in accordance with the present invention. The slow release formulations are typically implanted in the vicinity of the disease site.

Examples of sustained-release preparations include semipermeable matrices of solid hydrophobic polymers containing the polypeptide or immunoconjugate, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g., U.S. Pat. No. 3,773,919; copolymers of L-glutamic acid and γ ethyl-L-glutamate; non-degradable ethylene-vinyl acetate; degradable lactic acid-glycolic acid copolymers, such as the Lupron Depot (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

In some embodiments, the therapeutic agents and pharmaceutical compositions comprising the therapeutic agents described herein may be delivered to the central nervous system of the subject.

In some embodiments, the pharmaceutical compositions including one or more therapeutic agents can be provided and administered to a subject for the in vivo inhibition of Aggregatin induced amyloid β aggregation. The pharmaceutical compositions can be administered to any subject that can experience the beneficial effects of the therapeutic agents. Foremost among such animals are humans, although the present invention is not intended to be so limited, may be used to treat animals and patients with a neurodegenerative disease.

Pharmaceutical compositions for use in the methods described herein can have a therapeutically effective amount of the agent in a dosage in the range of 0.01 to 1,000 mg/kg of body weight of the subject, and more preferably in the range of from about 1 to 100 mg/kg of body weight of the patient. In certain embodiments, the pharmaceutical compositions for use in the methods of the present invention have a therapeutically effective amount of the agent in a dosage in the range of 1 to 10 mg/kg of body weight of the subject.

The overall dosage will be a therapeutically effective amount depending on several factors including the particular agent used, overall health of a subject, the subject's disease state, severity of the condition, the observation of improvements, and the formulation and route of administration of the selected agent(s). Determination of a therapeutically effective amount is within the capability of those skilled in the art. The exact formulation, route of administration and dosage can be chosen by the individual physician in view of the subject's condition.

The following examples is included to demonstrate different embodiments of the invention. It should be appreciated by those of skill in the art that the techniques disclosed in the examples, which follow represent techniques discovered by the inventor to function well in the practice of the claimed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the claims.

### Example

In this Example, we performed CPASSOC analysis of MRI measures and genetic datasets, and identified a possible link between FAM222A and AD-related regional brain atrophy. To understand its pathological role in AD, we investigated the protein encoded by FAM222A in patients with AD or transgenic mice for AD, and found its characteristic accumulation within the center of amyloid deposits. Further mechanistic study revealed that this protein could physically interact with Aβ and regulate Aβ aggregation and amyloid formation. Our results therefore identify a protein that likely plays an important role in amyloidosis, a finding providing perspective for AD pathogenesis.

### Methods

### Samples, Genotyping, and Imputation

Data was obtained from the ADNI database. The Illumina SNP genotyping data, demographic information, APOE genotype and baseline diagnosis information from 754 ADNI-1 participants, including 213 cognitive normal individual controls, 175 AD patients, and 366 patients with mild cognitive impairment (MCI) were downloaded from ADNI database. All participants provided written informed consent and study protocols were approved by participating sites' Institutional Review Board.

SNP genotyping of 620,901 markers on ADNI-1 participants were generated using Illumina BeadStudio 3.2 software from bead intensity data. All SNP genotypes are publicly available for download at the ADNI website. For genotype imputation analysis, only SNPs fulfilling the following criteria were included (1) per-SNP call rate≥0.98; (2) minor allele frequency (MAF)≥0.01; (3) P-value for Hardy-Weinberg equilibrium (HWE)≥10-6 in our sample set. Imputation was performed using the software MACH-ADMIX using the 1000 Genomes Project Phase 3 V.5 as a reference panel. We excluded SNPs with R2<0.3, MAF<0.01 and all INDELs from the imputed genotype data to obtain genotypes for 7,512,167 SNPs for subsequent association analyses.

### MRI Analysis and Extraction of Imaging Phenotypes

Baseline MRI T1 scans of ADNI1 participants were analyzed and generated using the 145 ROIs spanning the entire brain by using the Multi-atlas region Segmentation (MUSE) framework. In this framework, multiple atlases with semi-automatically extracted ground-truth ROI labels were first warped individually to the target image using non-linear registration methods. To fuse the ensemble into a final segmentation, they adopted a spatial adaptive weighted voting strategy, in which a local similarity term was used for ranking and weighting ground truth labels from different atlases and an image intensity based term was used for modulating the segmentations at the boundaries of the ROIs according to the intensity profile of the subject image. In validation experiments, the multi-atlas approach was shown to achieve significantly higher accuracy in comparison to single-atlas based segmentation. In this Example, we downloaded the volume measures of ROIs from ADNI.

### ROI-Wise Genome-Wide Association Analysis in ADNI1

Autosomal chromosome SNP associations for volumes from 145 ROIs spanning whole brain were assessed by linear regression under the assumption of an additive genetic model. All models were adjusted for age, gender, education, handedness and 3 principal components to control population stratification. The genomic control for 145 GWASs ranged between 0.98 to 1.02.

### Genetic Correlation Network Analysis of Brain ROIs in ADNI1

In multivariate quantitative genetics, a genetic correlation (r_{g}) is the proportion of variance that two traits share due to additive genetic effects, which estimates the degree of pleiotropy or causal overlap. The cross phenotype association analysis (CPASSOC) is a method proposed to integrate association evidence of multiple traits from multiple GWAS and detect cross-phenotype associations. Thus, CPASSOC analysis of genetic correlated AD-related brain imaging traits could improve power to identify genetic variants associated with multiple AD-imaging traits. To identify groups of highly genetic correlated ROIs, we used the estimated pairwise ROI genetic correlations to define the brain genetic correlation network. In this network, nodes are brain ROIs while edges are estimated genetic correlations between ROIs. To extract modules from this network, we adopted a weighted gene co-expression network analysis (WGCNA) framework and used the method of topological overlap matrix (TOM) elements in hierarchical clustering to identify modular structures. A flowchart for constructing a ROI genetic correlation network, extracting network modules and identifying genetic variants associated with modules using CPASSOC.

### Estimate Pairwise Genetic Correlations Among ROIs

Pairwise ROI genetic correlations were estimated by the technique of cross-trait LD score regression method using the GWAS summary statistics of ROIs. For 10,400 pairs among 145 ROIs, genetic correlations were not correctly estimated for 3,255 pairs because the estimated values were either ‘NA’, above 1 or below −1, which might be driven by the small sample size, and these pairs were then filtered out. However, this filter may reduce power to identify variants associated with ROIs. We observed high genetic correlations among the ROIs.

### Construct Genetic Correlation Network

We used the ROI genetic correlation matrix and power adjacency function to generate network adjacent matrix:

aᵢⱼ=|r_{gij}|^{β} (1)

while r_{gij} is the genetic correlation between nodes ROI i and ROI j, and aᵤ is the connection strength between two nodes.

To choose the parameter β and genetic correlation P-value threshold, we used the scale-free network model to construct an image network. The scale-free network assumes that most nodes in a network are sparsely connected with the exception of a few “hub” nodes that are densely connected with other nodes. In the scale-free network models, new connections are more likely to occur for those hub nodes with already-high connectivity, which meet biological criteria. We used the power law p(k)˜k^{-γ} to estimate the scale-free property, where k is the connectivity for each node and equals the number of its direct connections to other node. To generate the network, we assessed different power adjacency function parameter β=2, 4, 6 and 8 and filtered the genetic correlation with different r_{g} P-value thresholds of 0.5, 0.3, 0.2 and 0.1. For each P-value threshold, if the estimated genetic correlation P-value was larger than that, we set the genetic correlation to be 0. Using the four thresholds, we generated different networks for β=2, 4, 6 and 8 and accessed their corresponding scale-free topology using linear regression model fitting index R² between log₁₀(p(k)) and log₁₀(k) for all nodes. We observed that a P-value threshold of 0.2 with β=6 corresponded a network with the scale-free topology and had the largest R² of 0.61. The histogram of connectivity k and scale-free topology plots for networks with β=6 and different P-value threshold. Thus, we used the network adjacent matrix generated under this criterion for further analysis. In this network, 40 out of 145 ROIs had k equal to 0 and 105 ROIs were carried out in module identification analysis.

### Module Identification

We adopted the methods introduced by WGCNA framework⁴⁹ to identify network modules. The adjacent matrix was transformed into a topological overlap matrix (TOM) with element defined as:

$${w}_{ij} = \frac{{l}_{ij} + {a}_{ij}}{min \{ {k}_{i} , {k}_{j} \} + 1 - {a}_{ij}} with {l}_{ij} = {\sum }_{u} {a}_{iu} {a}_{uj} and {k}_{i} = {\sum }_{u} {a}_{ni}$$

TOM based dissimilarity measure was generated by:

dᵢⱼ^{w}=1−wᵢⱼ

This dissimilarity matrix was used as the input for average linkage hierarchical clustering. The hierarchical clustering grouped the closet ROIs and formed the branches to identify module. For the genetic correlation network, we identified 16 modules spanning the whole brain with the largest module containing 17 ROIs and the smallest containing 3 ROIs.

CPASSOC Analysis within Modules

We applied a CPASSOC package to combine association evidence of ROIs within each module. CPASSOC can integrate association evidence from summary statistics of multiple traits and improves power when variant is associated with at least one trait. CPASSOC provides two statistics, S_{Hom} and S_{Het} S_{Hom} is similar to the fixed effect meta-analysis method but accounting for the correlation of summary statistics among cohorts induced by potential overlapped or related samples. In brief, assuming we have summary statistical results of GWAS from J cohorts with K phenotypic traits. In each cohort, single SNP-trait association was analyzed for each trait separately. Let Tⱼₖ be a summary statistic for a SNP, j^{th} cohort and k^{th} trait. Let T=(T₁₁, . . . , T_{J1}, . . . , T_{1K}, . . . , T_{JK})^{T} represents a vector of test statistics for testing the association of a SNP with K traits. We used a Wald test statistic

$${T}_{j k} = \frac{{\hat{β}}_{j k}}{{\hat{s}}_{j k}} ,$$

where \hat{β}ⱼₖ and ŝⱼₖ are the estimated coefficient and corresponding standard error for the k^{th} trait in the j^{th} cohort. S_{Hom} is then defined as:

$${S}_{H o m} = \frac{{{e}^{T} ( R W )}^{- 1} {T ( {{e}^{T} ( R W )}^{- 1} T )}^{T}}{{{e}^{T} ( WRW )}^{- 1} e} ,$$

which follows a χ² distribution with one degree of freedom, where e^{T}=(1, . . . , 1) has length J×K and W is a diagonal matrix of weights for the individual test statistics. We used the sample sizes for the weights, wⱼₖ=\sqrt{nⱼ}, nⱼ is sample size of the j^{th} cohort.

To further allow for different effect directions of a variant for different traits in different cohorts, we define S_{Het}, we first let:

$$S ( τ ) = \frac{{{{e}^{T} ( R ( τ ) W ( τ ) )}^{- 1} T ( τ ) {( R ( τ ) W ( τ ) )}^{- 1} T ( τ ) )}^{T}}{{e}^{T} {W ( τ )}^{- 1} {R ( τ )}^{- 1} {W ( τ )}^{- 1} e} ,$$

where T(τ) is the sub-vector of T satisfying |Tⱼₖ|>τ for a given τ>0, and R(τ) is a sub-matrix of R representing the correlation matrix, and W(τ) be the diagonal submatrix of W, corresponding to T(τ). Here we let wⱼₖ=\sqrt{nⱼ}×sign(Tⱼₖ). Then the test statistic is S_{Het}=max_{τ>0}S(τ).

The asymptotic distribution of S_{Het} does not follow a standard distribution but can be evaluated using simulation. S_{Het} is an extension of S_{Hom} but power can be improved when the genetic effect sizes vary for different traits. The distribution of S_{Het} under the null hypothesis can be obtained through simulations or approximated by an estimated beta distribution.

We applied both S_{Hom} and S_{Het} to combine summary statistics for ROIs within each module. The CPASSOC analysis of multiple genetic correlated traits in identified module would allow us to identify variants that are likely to be missed by conventional GWAS of single trait and reduce the multiple comparison burden in the genetic analysis of hundreds of neuroimaging traits. Finally, we identified 15 loci with CPASSOC test P-value less than 1×10⁻⁷ in nine modules. Importantly, three previously reported AD associated SNPs, rs429358, rs2075650 and rs439401 and the FAM222A SNP rs117028417 were exclusively found in one module.

### Genetic Analysis of AV-45 PET Imaging

¹⁸F-Florbetapir (AV-45) PET imaging was performed at baseline and 2-year follow-up for participants enrolled in the ADNI GO and two phases. UC Berkeley extracted weighted AV-45 standardized uptake value ratio (SUVR) means for four main cortical regions: frontal, anterior, and posterior cingulate, lateral parietal and lateral temporal regions (version 2019.4.12) for ADNI-GO2 participants. They also calculated composite SUVR for cortical which is weighted SUVR mean in frontal, cingulate, parietal and temporal regions. These data can be downloaded from the ADNI database. We used the SUVR mean of composite region including whole cerebellum, pons/brainstem and eroded white matter as reference. Mean AV-45 SUVR of frontal, cingulate, lateral parietal, lateral temporal and composite cortical relative to the reference were calculated. The annual percent change in SUVR means at 2-year follow-up compared to baseline was used as the main quantitative phenotype for genetic analysis. The annual percent changes in AV-45 SUVR for all five brain regions were approximately normally distributed. We collected 369 individuals with both SUVR measures for baseline and 2-year follow-up and whole-genome sequencing data. The samples included 120 healthy people, 26 people with AD, 64 people with late mild cognitive impairment (LMCI) and 159 people with early mild cognitive impairment (EMCI) diagnosed at baseline.

WGS data from 817 ADNI participants were downloaded from the ADNI dataset. WGS was performed using blood-derived genomic DNA samples and sequenced on the Illumina HiSeq2000 using paired-end read chemistry and read lengths of 100 bp at 30-40× coverage. As previously described using Broad GATK and BWA-mem, reads were mapped and aligned to the human genome (build 37), then variants were called.

For single SNP association test, association test of SNP rs117028417 with phenotypes were performed using linear regression under an additive genetic model in PLINK. Baseline age and gender were included as covariates. For gene based association test, we extracted 8 and 6 functional coding variants defined as missense, in frame deletion/insertion, stop gained/lost, start gained/lost, splice acceptor/donor, or initiator/start codon for FAM222A and TRPV4 respectively. All of those variants are rare with minor allele frequency (MAF)<0.01 in ADNI samples. Gene-based association tests were performed using burden and SKAT, adjusting age and sex as covariates.

### Genetic Analysis of CSF Aβ and Tau

Collection and processing of ADNI CSF samples was described in the ADNI procedures manual. We downloaded UPENNBIOMKs dataset.csv file from ADNI website. We collected 617 individuals with both CSF Aβ42, tTau and pTau at baseline level and WGS data. For baseline data, since raw CSF biomarkers were skewed or bimodal skewed distributed, rank normal transformations were conducted for each biomarker separately. To conduct CSF biomarkers longitudinal change genetic association, we collected 274 individuals with both baseline and 24-month follow-up CSF biomarkers and WGS data. The CSF biomarkers raw data at baseline and 2-year follow-up in 218 individuals were used to calculate annual changes in Aβ42, tTau and pTau separately. The annual changes of three CSF biomarkers were approximately normally distributed.

Association test of SNP rs117028417 with phenotypes were performed using linear regression under an additive genetic model in PLINK. Baseline age and sex were included as covariates. We extracted 8 and 15 coding variants defined as missense, in frame deletion/insertion, stop gained/lost, start gained/lost, splice acceptor/donor, or initiator/start codon for FAM222A and TRPV4 respectively. All of those variants are rare with minor allele frequency (MAF)<0.01 in ADNI samples. Gene-based association tests were performed using burden and SKAT, adjusting age and sex as covariates.

Analysis of FAM222A mRNA in AD

The development of the Mount Sinai Brain Bank (MSBB) cohort has been described. MSBB is a large AD cohort and now holds over 2,040 well-characterized human brains. The datasets we used assessed a total of 125 human brains which was assembled after applying stringent inclusion/exclusion criteria and represents the full spectrum of cognitive and neuropathological disease severity. We downloaded the normalized microarray data of MSBB Array Tissue Panel Study from the Synapse. The RNA samples from 19 brain regions isolated from 125 MSBB specimens were collected and profiled on the Affymetrix 133AB and Affymetrix 133Plus2 platforms. RNA quality was assessed using a combination of a 260/280 ratio derived from resolution electrophoresis system (LabChip™, Agilent Technologies, Palo Alto, Calif., USA) and 3′-5′ hybridization ratios for GAPDH probes. Not all brain regions for all subjects were available for analysis. There was an approximately 60 samples (40 AD, 20 controls) per brain region available for analysis. The array probes were annotated according to the Ensemble version 72 (genome build GRCh37) using the R/Biomart library. The raw microarray data were quantile normalized with all probe sets on the arrays using RMA method implemented in the R/Bioconductor package affy (v1.44) with the default parameters. The data were then corrected for covariates including sex, postmortem interval (PMI), pH and race using a linear regression model. The FAM222A gene expression data was identified by probe set 226487_at. The processed FAM222A mRNA level means for groups of AD and control were compared using two-sided Welch t-test using R.

### Association Analysis of FAM222A DNA Methylation

We downloaded two datasets, E-GEOD-45775 and E-GEOD-76105, with DNA methylation profiling from the European Bioinformatics Institute (EMBL-EBI) ArrayExpress website. Samples of dataset E-GEOD-45775 included 5 controls, 5 AD Braak stage I-II and 5 AD Braak stage V-VI. The methylation values were adjusted and normalized using BeadStudio software v3.2 to obtain normalized beta and average Beta detect P-value. The array used the HumanMethylation27_270596 v.1.2 design and one methylation site cg01335367 was identified located on chr12:109734355-109734404 (GRCh38.p12), associated with FAM222A. We analyzed the association between methylation in cg01335367 with AD using logistic regression and adjusted for sex. We also performed one-way analysis of variance (ANOVA) to determine differences between methylation levels of control and different Alzheimer Braak stage groups. Study EGEOD-70615 investigated DNA methylation profiling in the superior temporal gyrus (STG). Samples included 34 AD and 34 non-demented controls, which had 52 European, 8 Hispanic, 6 African, 1 Asian Americans and 1 unknown. The Beta values from the probes were quantile normalized using lumi package in R. We performed association analysis in 52 European Americans only. The association between methylation in those sites with AD were analyzed using logistic regression model adjusting age, gender and estimated cellular proportions (neuronal vs. glial).

### Mice and Human Tissues

Mouse surgery and procedures were performed according to the NIH guidelines and were approved by the Institutional Animal Care and Use Committee (IACUC) at Case Western Reserve University. 5×FAD transgenic mice (B6.Cg-Tg(APPSwFlLon, PSEN1*M146L*L286V) 6799Vas/Mmjax, JAX #008730) were purchased from the Jackson Laboratory. The use of all human tissue samples was approved by the University Hospitals Institutional Review Board (IRB) for human investigation at University Hospitals Case Medical Center at Cleveland. Human brain tissues obtained postmortemly from University Hospitals of Cleveland were fixed, and 6-μm-thick consecutive sections were prepared.

### Immunocytochemistry, Immunofluorescence and Immunoblot

Immunocytochemistry was performed by the peroxidase anti-peroxidase protocol. Taken briefly, paraffin embedded brain tissue sections were first deparaffinized in xylene and rehydration in graded ethanol and incubated in Tris Buffered Saline (TBS, 50 mM Tris-HCl and 150 mM NaCl, pH 7.6) for 10 min before antigen retrieval in 1× Immuno/DNA retriever with citrate (BioSB, Santa Barbara, Calif.) under pressure using BioSB's TintoRetriever pressure cooker. Sections were rinsed with distilled H₂O, and blocked with 10% normal goat serum (NGS) in TBS at room temperature (RT) for 30 min. Tissue sections were further incubated with primary antibodies in TBS containing 1% NGS overnight at 4° C., and immunostained by the peroxidaseantiperoxidase based method. For double Immunofluorescence staining, paraffin embedded tissue sections were deparaffinized in xylene and re-hydrated in graded ethanol without H₂O₂ incubation as described above. The sections were incubated in phosphate buffered saline (PBS) at RT for 10 min followed by block with 10% NGS in PBS for 45 min at RT. The sections were incubated with primary antibodies in PBS containing 1% NGS overnight at 4° C. After being washed with 1% NGS in PBS for 10 min, the sections were incubated in 10% NGS for 10 min and followed by three quick washes with 1% NGS in PBS. Then, the sections were incubated with Alexa Fluor 488 or 568 dye labeled secondary antibodies (1:300, Invitrogen, Carlsbad, Calif.) for 2 h at RT in dark, washed three times with PBS, stained with DAPI, washed again with PBS for three times, and finally mounted with Fluoromount-G mounting medium (Southern Biotech, Birmingham, Ala.). For thioflavin-S staining, slides were incubated with 1% thioflavin-S (Santa Cruz Biotechnology, Dallas, Tex.) for 8 min, washed 2 times with 80% ethanol, and 1 time with 95% ethanol and PBS, then stained with DAPI. For immunoblot, human or mice tissue samples were all lysed with TBS plus 1 mM phenylmethylsulfonyl fluoride (PMSF) (Millipore, Burlington, Mass.), protease inhibitor cocktail (Sigma Aldrich, St. Louis, Mo.) and phosphatase inhibitor cocktail (Sigma Aldrich, St. Louis, Mo.). Equal amounts of total protein extract were resolved by SDS-PAGE and transferred to Immobilon-P (Millipore, Burlington, Mass.). Following blocking with 10% nonfat dry milk, primary and secondary antibodies were applied and the blots developed with Immobilon Western Chemiluminescent HRP Substrate (Millipore, Burlington, Mass.). Images were taken by ChemiDoc Touch Imager (Biorad, Hercules, Calif.). The dilution of antibodies used for IF or IHC. 4G8 (BioLegend, SIG-39220; IF, 1:1000), 6E10 (BioLegend, 803001; IF and IHC, 1:1000), 82E1 (IBL, 10323; IF, 1:1000), Aggregatin (Abcam, ab122626; IF/IHC, 1:100), Aggregatin (LifeSpan BioSciences, LS-C170630; IHC, 1:1000), Aggregatin (Aviva Systems Biology, ARP69038_P050; IHC, 1:1000), Flag (Sigma Aldrich, F1804; IF/IHC, 1:1000), Flag (Thermo Fisher, PA1-984B; IHC, 1:200), Flag (Cell Signaling Technology, 2368; IHC, 1:200), Flag-HRP (Proteintech, HRP-66008; IHC, 1:1000), GFP (Abcam, ab32146; IHC, 1:500), Myc (Thermo Fisher, MA1-21316; IHC, 1:1000), Myc (Cell Signaling Technology, 2276; IHC, 1:500), and Nu4 (Klein lab, IF/IHC, 1:2000). All uncropped and unprocessed blots are provided in the Source Data file (Source Data for Statistics and Blots).

### Expression Vectors and Recombinant Proteins

pcDNA3.1(+) (Invitrogen, Carlsbad, Calif.) plasmid was modified to express recombinant proteins to express recombinant proteins containing a 4×Flag-Twin-Strep-tag at their N-terminal. The cDNA of full length or truncated human Aggregatin were inserted into the modified pcDNA3.1(+) plasmid. Eight micrograms plasmid was used to transfect one 10 cm dish of Lenti-293T cells with TransIT®-293 Transfection Reagent (Mirus, Madison, Wis.). Cells were collected at 24 h after transfection and lysed by lysis buffer (100 mM Tris-HCl, 150 mM NaCl, 1 mM EDTA and 1% NP40, pH 8.0) containing 1 mM PMSF (Millipore, Burlington, Mass.), protease inhibitor cocktail (Sigma Aldrich, St. Louis, Mo.) and phosphatase inhibitor cocktail (Sigma Aldrich, St. Louis, Mo.). The lysate was centrifuged at 14,000 g for 15 min at 4° C. Supernatant was incubated with Mag-Strep type3 XT beads (IBA Lifesciences, Goettingen, Germany) overnight at 4° C. Beads were washed three times with lysis buffer, and eluted with BXT buffer (IBA Lifesciences, Goettingen, Germany) overnight at 4° C. At last, the eluted recombinant proteins were subjected to dialysis using 10 kD Slide-A-Lyzer™ Dialysis Cassettes (Thermo Fisher Scientific, Waltham, Mass.), concentration with 10 kD Spin Column (Abcam, Cambridge, Mass.) and purification by size-exclusion chromatography.

### Stereotaxic Injection and ICV Infusion

Mice surgery were performed according to the NIH guidelines and were approved by the Institutional Animal Care and Use Committee (IACUC) at Case Western Reserve University. All AAVs with 1013 genome copies per mL (GC per mL) were obtained from Vigene Biosciences (Jinan, China). For stereotaxic injection, mice were anesthetized with isoflurane and immobilized using the stereotactic frame equipped with a heating blanket to maintain body temperature throughout the procedure. After hair removal and the cleaning of the shaved area with betadine and alcohol, mice were injected with bupivacaine/lidocaine and a small incision was made to expose the skull surface. Two small holes were drilled in the skull (relative to bregma: anteroposterior −2.1 mm, medial lateral ±2 mm; Note that ± is the plus-minus sign throughout this study) followed by injection of 2 μl AAVs using Hamilton syringes into the hippocampal CA1 at dorsal ventral −1.45 mm. Injection speed was pump controlled at 0.2 μl per min. The needle was left in place for 5 min before it was slowly withdrawn. For ICV infusion, the mini-osmotic pump (Model 1004, Alzet, Cupertino, Calif.; flow rate of 0.11 μl per hour, 28 days) and brain infusion cannula attached with 2.5-3 cm catheter tubes (Brain infusion kit 3, Alzet, Cupertino, Calif.) were filled with recombinant protein in artificial cerebrospinal fluid (aCSF), followed by pump incubation in aCSF at 37° C. for 48 h according to the manufacturer's instructions. For implant surgery, a hole was drilled in the skull (relative to bregma: anteroposterior −0.5 mm, medial lateral 0.75 mm). The cannula was positioned on the skull with the needle plug 2.5 mm into the ventricle. The cannula was fixed and secured by cyanoacrylate glue.

### Behavioral Tests

Mice behavioral tests were also performed according to the NIH guidelines and were approved by the Institutional Animal Care and Use Committee (IACUC) at Case Western Reserve University. The Barnes maze consisted of a white acrylic circular disk 92 cm in diameter with 20 equally spaced holes (5 cm in diameter) located 2 cm from the edge of the disk. The maze was illuminated by two 60 W lamps to provide an aversive, bright disk surface. An acrylic escape box (7×7×5 cm) could be fitted under any of the holes in the maze. The maze was raised 30 cm from the floor and rested on a pedestal that enabled it to be rotated 360° on a horizontal plane. An acrylic start bin with 15 cm diameter and 15 cm height was used. Trials were recorded using a webcam and analyzed by video tracking software (EthoVision XT, Noldus, Leesburg, Va.). Each trial began with the start bin positioned in the center of the maze with the mouse placed inside. The mouse remained in the start bin for 30 s, providing a standard starting context for each trial and ensuring that initial orientation of the mouse in the maze varied randomly from trial to trial. Each mouse was allowed to explore the maze freely for 2 min. After the mouse entered the escape hole, the mouse was left in the escape box for 90 s before being returned to its home cage. If the mouse did not enter the escape box within 120 s, it was gently picked up by the experimenter and placed over the target hole and allowed to enter the escape box. After each trial, the maze and escape box were cleaned carefully with a 10% alcohol solution to dissipate odor cues and provide a standard olfactory context. Five training sessions consisting of two trials each were run on subsequent days and escape latencies were measured. For Y maze test, mice were placed in a Plexiglas Y maze (with arms 60 cm in length) and allowed to explore the maze freely for 10 min. When put in the Y maze, the mice were recorded using the ANY-maze tracking system, and the time and frequency in the spontaneous alteration ratio were counted automatically. All tests were performed at the Case Behavior Core, with the investigator blinded to mouse genotype.

### Plaque Isolation

Amyloid plaque cores were isolated. Briefly, whole mouse brain or human brain gray matters were homogenized, boiled in lysis buffer (2% SDS, 50 mM Tris-HCl pH 7.5, 50 mM DTT), and centrifuged at 100,000 g for 1 h at 10° C. The pellet was solubilized in fraction buffer (1% SDS, 50 mM Tris-HCl pH 7.5, 50 mM DTT) and centrifuged at 100,000 g for 1 h at 10° C. The pellet was further suspended in fraction buffer and loaded on top of a discontinuous sucrose gradient (1.0, 1.2, 1.4 and 2.0M sucrose in 50 mM Tris pH 7.5 containing 1% SDS), centrifuged at 220,000 g for 20 h at 10° C. and fractionated into sixteen fractions (300 μl per fraction). Plaque-core-enriched fraction #13 were further diluted in fraction buffer and centrifuged at 220,000 g for 2 h at 10° C. The resulting pellet was dissolved in 70% formic acid and subsequently dried using a SpeedVac system. Solubilized proteins were further resuspended in 1×SDS sample buffer with 8M Urea.

### Aβ Preparation, Pull-Down, and Co-Sedimentation Assay

Synthetic human Aβ1-42 and Aβ1-40 peptides (GL Biochem, Shanghai) were dissolved in hydroxylfluro-isopro-panol (HFIP) and subsequently dried using a SpeedVac system. Both Aβ1-42 and Aβ1-40 monomers were prepared by dissolving the lyophilized Aβ in dimethyl sulfoxide (DMSO) at 5 mM, sonicated for 10 min and diluted in PBS buffer (NaCl 137 mM, KCl 2.7 mM, Na2HPO4 10 mM, KH2PO4 1.8 mM, pH 7.4) to different concentrations. Aβ1-42 oligomers were prepared in DMSO/PBS and oligomerized by incubation at 4° C. for 24 or 48 h. Monomeric or oligomer Aβ1-40 (100 μM) and Aβ1-42 solutions (50 μM) supplemented with or without rAggregatin bound Strevdin-avdin beads were incubated in IP buffer (NaCl 300 mM, KCl 2.7 mM, Na2HPO4 10 mM, KH2PO4 1.8 mM, pH7.4) at RT with shaking for 2 h. After 4 times wash with IP buffer, beads were eluted by 1×SDS sample buffer (32.9 mM Tris-HCl pH6.8, 13% Glycerol, 1% SDS and 0.005% bromophenol blue) and analyzed by 10-20% SDS/Tricine protein gels (Invitrogen, Carlsbad, Calif.). For Aβ1-42 oligomer formation and co-sedimentation assay, HFIP dissolved synthetic Aβ1-42 peptides were solubilized in 30 mM NaOH to a final concentration of 100 μM, diluted to 2.5 μM in PBS and incubated with and without 30 nM rAggregatin at 37° C. for different time points. After 10-minute centrifuge at 14,000 g, pellets and supernatants were collected and analyzed by 10-20% SDS/Tricine protein gels (Invitrogen, Carlsbad, Calif.).

### Dynamic Light Scattering

Dynamic light scattering (DLS) experiments were carried out with DynaPro™ instrument from Wyatt technology with a wavelength of 633 nm and a scattering angle of 173°. The measurements of Aggregatin or Aggregatin 461-80 at 100 nM were performed at 25° C. after 2 min equilibration with correlation times defined on 10 s per run with 30 runs for each measurement. The results were plotted as intensity of distribution (%) of particles versus hydrodynamic radius (nm).

### Circular Dichroisms

The spectra were recorded over a wavelength range of 260-190 nm with standard sensitivity at the 50 nm per min scan speed with 1-nm resolution and 1-s time constant at room temperature using a spectropolarimeter (Jasco J-815). All the proteins were dissolved in phosphate buffer (pH8.0). The final concentration of all samples was 1 μM. The secondary structure content was calculated from the Circular dichroisms (CD) spectra using the online software K2D3.

### Surface Plasmon Resonance

Surface plasmon resonance (SPR) was determined using BIAcore3000 (GE Healthcare Life Sciences, Pittsburgh, Pa.). rAggregatin (0.1 mg per ml) was immobilized on the CMS sensor surface (GE Healthcare Life Sciences, Pittsburgh, Pa.) in 10 mM acetate buffer (pH=4.5). Running buffer was 1% DMSO in PBS-P buffer (0.02M phosphate, 2.7 mM KCl, 137 mM NaCl and 0.05% Tween 20). Binding of a dilution series comprising Aβ1-42 monomers to rAggregatin was analyzed and fitted to the 1:1 binding model using BIAevaluation software (GE Healthcare Life Sciences, Pittsburgh, Pa.).

### Solid Phase Binding Assay

rAggregatin was coated onto Nunc MaxiSorp 96-well plates (Thermo Fisher Scientific, Waltham, Mass.) at 0.1 μg per well in PBS at 4° C. overnight. After blocking in 1% BSA in PBS for 2 h at RT, Aβ1-42 at 6.25, 12.5, 25, 50, 100, or 200 nM or Aβ1-40 at 0.5, 1, 2, 4, or 8, or 16 μM monomers were added to the plates at 4° C. overnight. Plates were washed with PBS 4 times and incubated with 6E10 antibody at 4° C. overnight, followed by 4 times PBS wash and development in TMB solution (Thermo Fisher Scientific, Waltham, Mass.). The reaction was stopped by sulfuric acid and assessed using a Synergy H1 microplate reader (BioTek, Winooski, Vt.). Likewise, 0.2 μg Aβ1-42 or Aβ1-40 monomers were immobilized on plates and incubated with 3.125, 6.25, 12.5, 25, 50, or 100 nM rAggregatin. Bound rAggregatin were detected by an anti-Flag antibody and developed in TMB solution as described above.

### ThT Fluorescence Assay

HFIP treated Aβ1-40 or Aβ1-42 peptides were solubilized in 30 mM NaOH to a final concentration of 400 μM, sonicated for 5 min in a water bath and stored at −80° C. until further use. To monitor Aβ1-40 and Aβ1-42 fibrillization, a ThT assay was performed according previous studies. Briefly, a stock solution of Aβ was diluted to in PBS with 20 μM ThT. Then rAggregatin were added at desired concentrations to the final volume of 100 μl. All samples were transferred to a black 96-well nonbinding Surface microplate with clear bottom (Corning, Corning, N.Y.), and sealed with a polyester-based sealing film (Corning, Corning, N.Y.). Samples were incubated at 37° C. with stirring. Real-time ThT fluorescence was measured every 5 min for at least 12 h at the excitation and emission wavelengths of 446 nm and 482 nm respectively by a Synergy H1 microplate reader (BioTek, Winooski, Vt.).

### Aβ1-42 Aggregates Stained by Thio-S

To evaluate Aβ aggregates formed in vitro, rAggregatin (30 nM) and 2.5 μM Aβ in PBS were incubated at 37° C. for 4 weeks. 20 μl of protein solution were applied to the glass slides and completely air dry for 30 min. After washing with PBS, the samples were stained by 1% Thio-S for 10 min. The 3D confocal images were analyzed by using Imaris (Bitplane, Concord, Mass.) and the structure surface were extracted by using the SURFACE tools following the manufacturer's instructions.

### Negative Electric Microscopy

HFIP dissolved synthetic Aβ1-42 peptides were solubilized in 30 mM NaOH to a final concentration of 100 μM. Then diluted to 2.5 μM in PBS and incubated with and without 30 nM rAggregatin at 37° C. Immediately following the indicated incubation time, 20 μl of protein solution were applied to the support surface of the grids, which were autoclaved by UV irradiation overnight. The grids were washed with 20 μl droplets of water 4 times, followed by a 20 μL droplet of uranyl acetate solution, then examined in an FEI Tecnai Spirit (T12) with a Gatan US4000 4kx4k CCD.

### Total Aβ Measurement by ELISA

Brains were homogenized in TBS Buffer (50 mM Tris-HCl and 150 mM NaCl, pH 7.6) containing 1 mM PMSF (Millipore, Burlington, Mass.), protease inhibitor cocktail (Sigma Aldrich, St. Louis, Mo.) and phosphatase inhibitor cocktail (Sigma Aldrich, St. Louis, Mo.). Total protein concentrations were determined using the BCA kit (Thermo Fisher Scientific, Waltham, Mass.). ELISA of total Aβ was carried out in 96-well high-binding microtiter plates. Monoclonal antibody 6E10 raised against residues Aβ1-16 was used as a capture antibody (diluted in PBS pH 7.4) and incubated over night at 4° C. in a humid chamber. After removal of the capture antibody, the plate surface was blocking with 1% BSA for 1.5 h. After washing with PBS, 0.5 μg total protein were added and incubated at 4° C. overnight. Monoclonal antibody MOAB-2 coupled to horseradish peroxidase diluted in PBS were used as secondary antibodies and again incubated over night at 4° C. After three times washing with PBS, 100 μl of TMB ELISA peroxidase substrate (Thermo Fisher Scientific, Waltham, Mass.) was added and incubated for 1-10 min at RT in darkness. The reaction was stopped with 100 μl 2M H2SO4 and absorbance was measured in a microplate reader at 450 nm. For generation of standard curves, synthetic Aβ1-42 peptides freshly dissolved in DMSO from 1 ng per μL to 10 pg per μL.

### Isolation of Exosomes

Lenti-293T cells were transfected with empty vector or pCDNA-4×Flag-Aggregatin using TransIT®-293 Transfection Reagent (Mirus, Madison, Wis.). Twenty-four hours after transfection, cells were cultured in the DMEM medium supplemented with exosome-free FBS. Forty-eight hours later, the cell culture medium was collected and centrifuged at 300 g for 15 min to remove cells and debris. The supernatant was further filtered through a 0.22 μm filter and centrifuged at 100,000 g for 2 h at 4° C. The pellets enriched with exosomes were resuspended in the lysis buffer (100 mM Tris-HCl, 150 mM NaCl, 1 mM EDTA and 1% NP40, pH 8.0) containing 1 mM PMSF (Millipore, Burlington, Mass.), protease inhibitor cocktail (Sigma Aldrich, St. Louis, Mo.), and phosphatase inhibitor cocktail (Sigma Aldrich, St. Louis, Mo.) followed by immunoblot analysis.

### Confocal Microscopy and Image Analysis

All fluorescence images were imaged on a Leica TCS SP8 gSTED confocal microscopy (Leica Microsystems, Buffalo Grove, Ill.) equipped with a motorized super Z galvo stage, two PMTs, three Hyd SP GaAsP detectors for gated imaging, and the AOBS system lasers including a 405 nm, Argon (458, 476, 488, 496, 514 nm), a tunable white light (470 to 670 nm), and a 592 nm STED depletion laser. Series of confocal images with optical thickness of 300 nm were collected using the ×100 oil objective. All 3D confocal images of plaque were reconstructed using Imaris (Bitplane, Concord, Mass.) after background subtraction. Quantification of Aggregatin foci in plaques and measurement of plaque load and size were performed with open-source image analysis programs WCIF ImageJ (developed by W. Rasband).

### Statistical Analysis

Statistical analysis was done with one-way analysis of variance (ANOVA) followed by Tukey's multiple comparison test or student-t-test using GraphPad Prism (GraphPad, CA). Data are means±SEM. p<0.05 was considered to be statistically significant.

FAM222A-Encoded Protein Accumulates within Amyloid Plaques

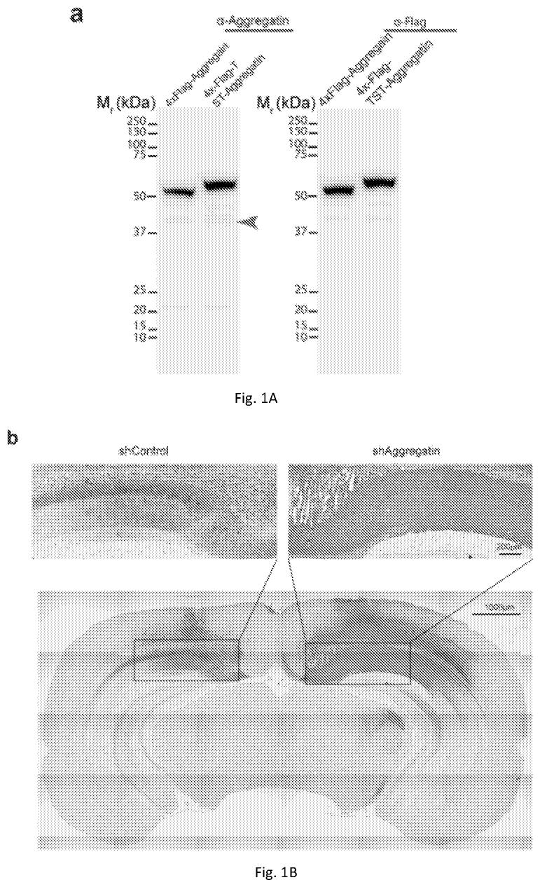

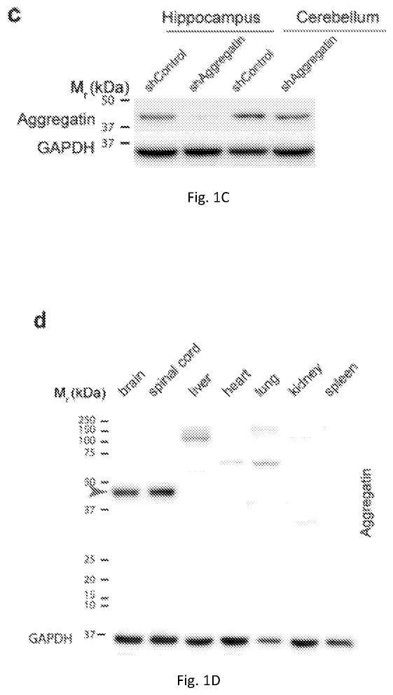

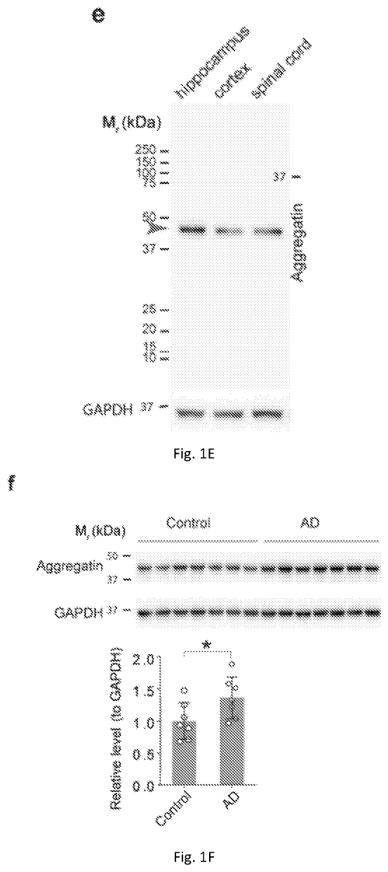

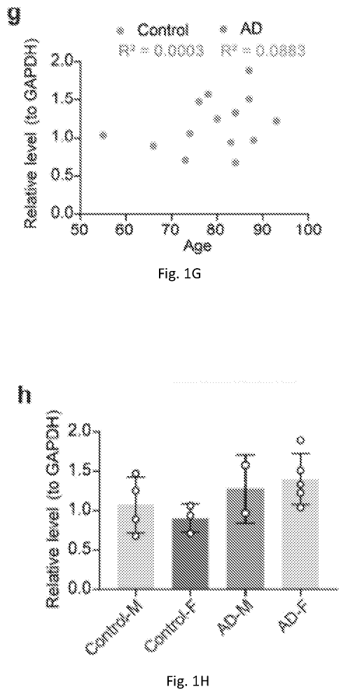

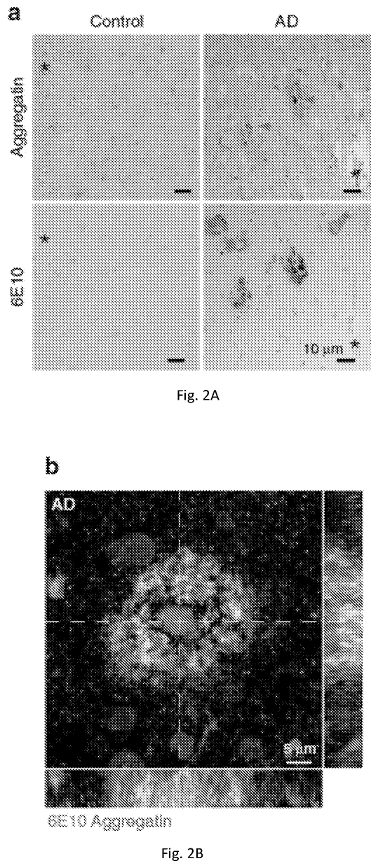

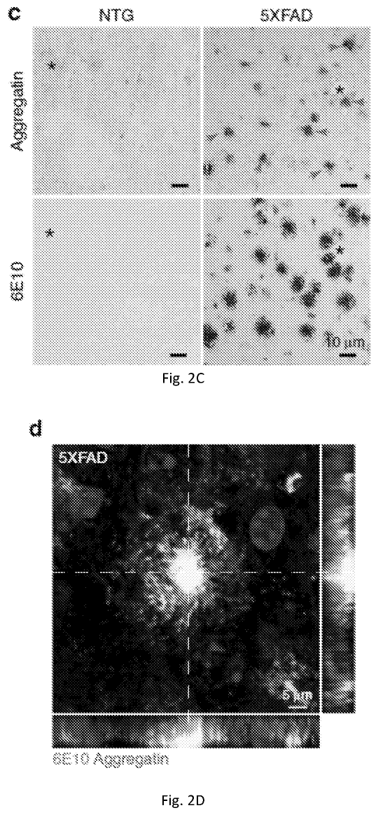

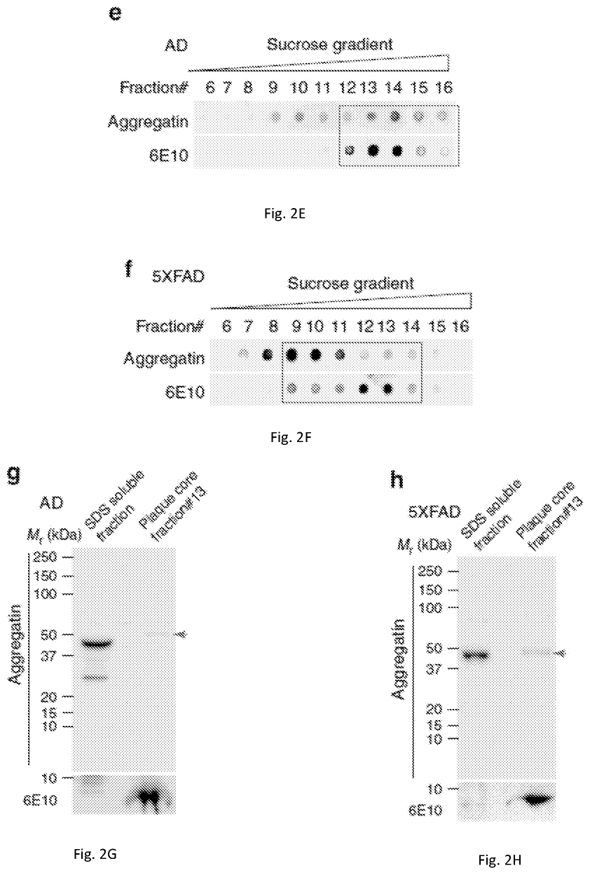

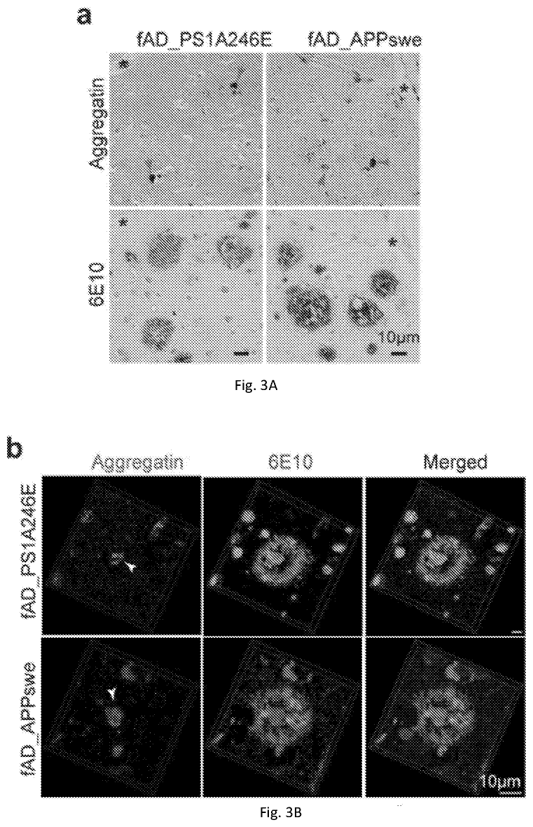

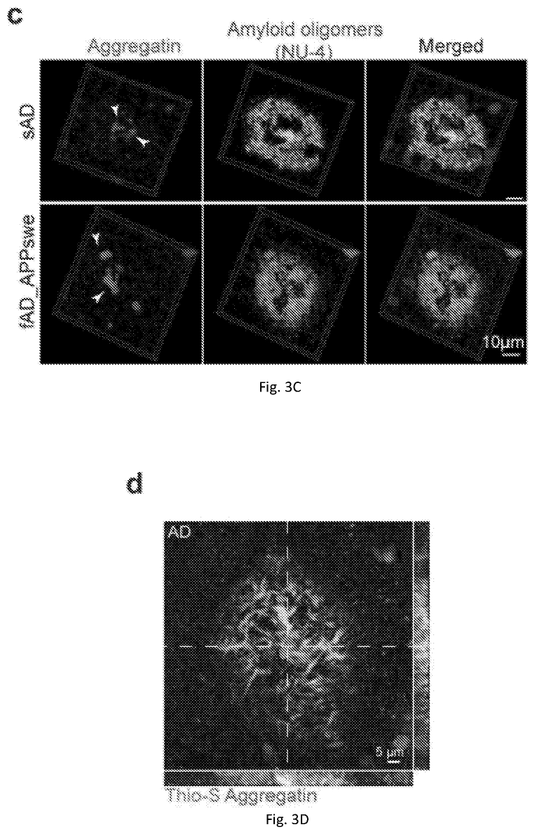

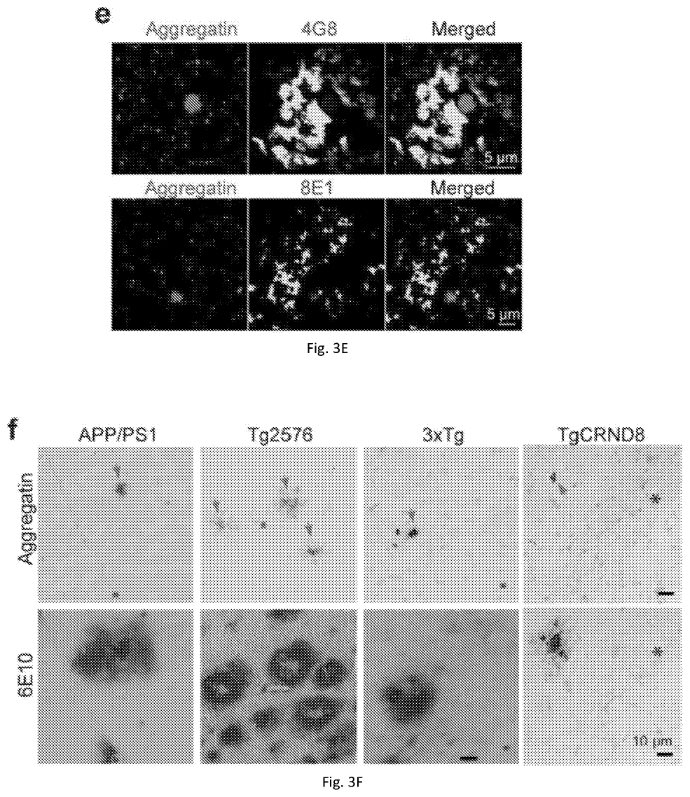

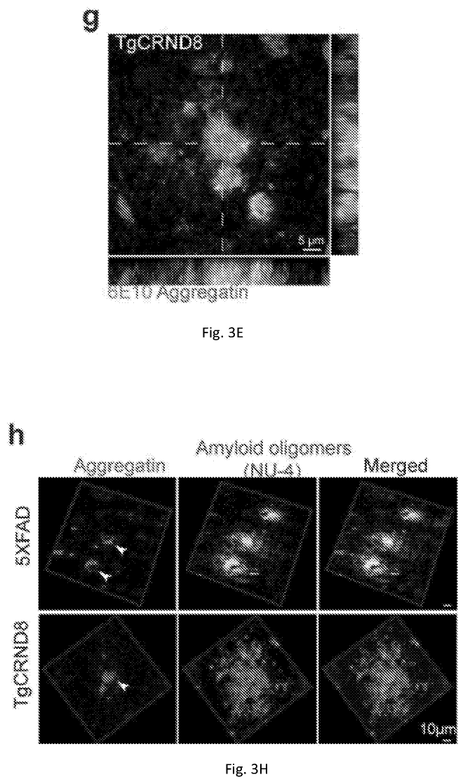

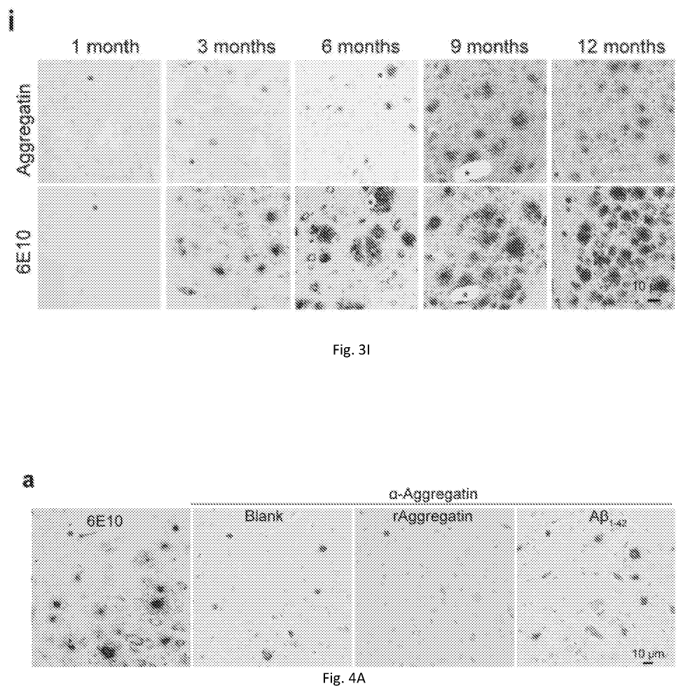

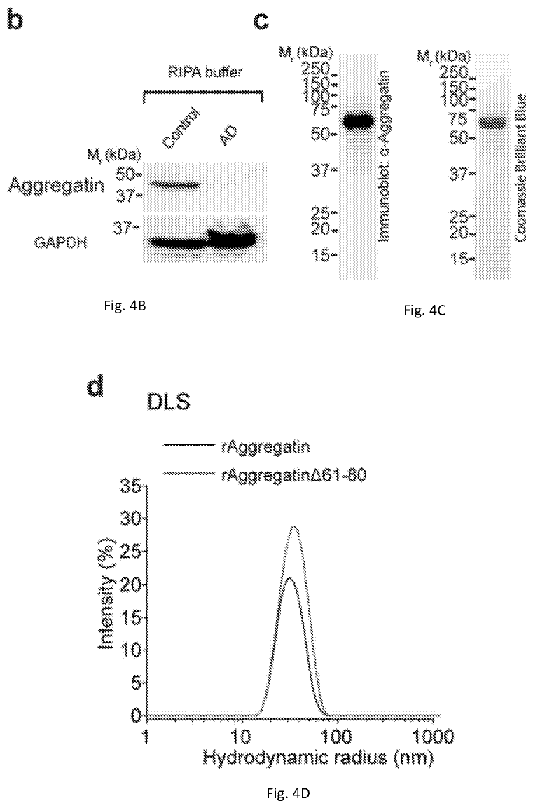

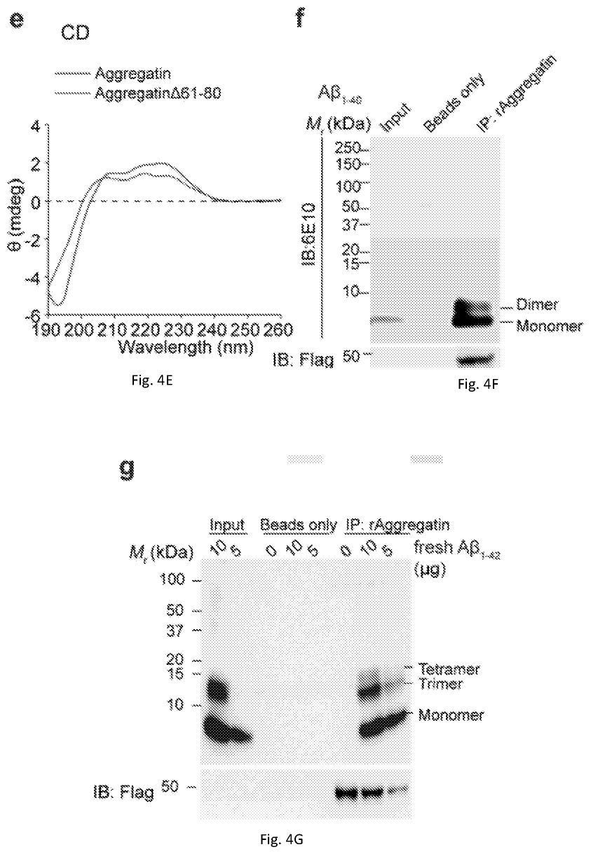

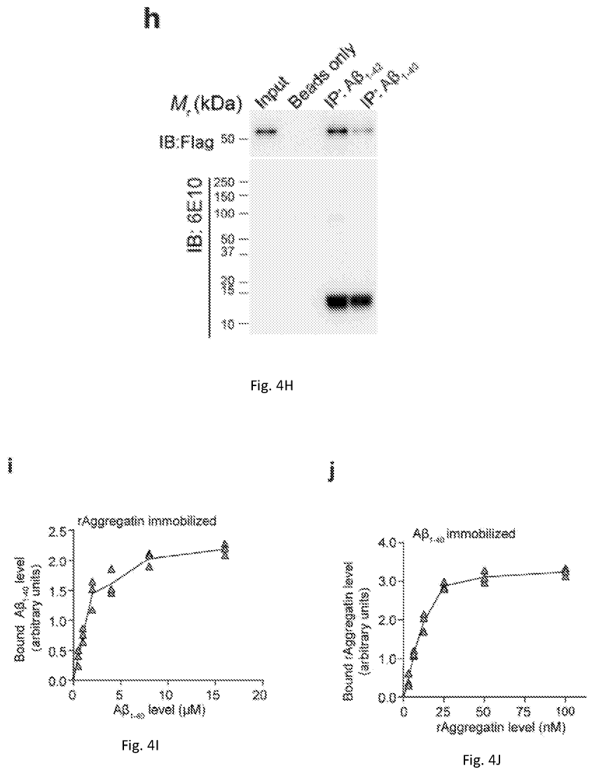

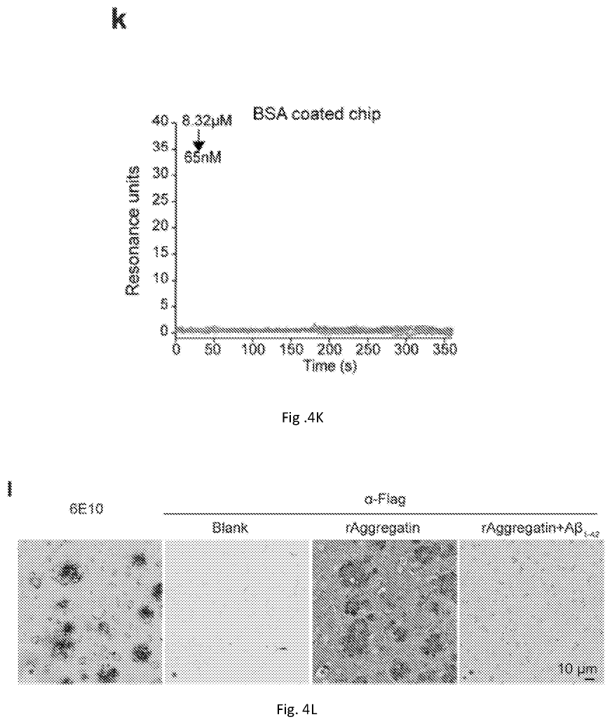

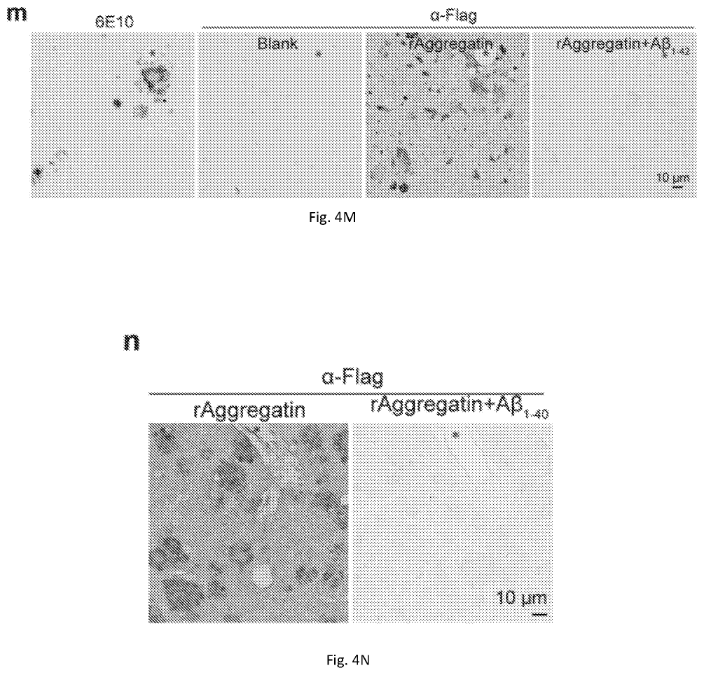

To elucidate the possible pathological role of FAM222A in AD, we carried out experimental validation to focus on its encoded protein, which we designated as Aggregatin. Aggregatin consists of 452 amino acids with a predicted molecular weight of 47 kD, and has not yet been characterized. Using a well-characterized specific antibody against Aggregatin (FIGS. 1A-E), Aggregatin was found predominantly expressed in the central nervous system (CNS) including both the brain and the spinal cord, but not in other tissues such as heart, spleen, lung, kidney, or liver in mice or humans (FIGS. 4D, E). There was a slight increase in the expression of Aggregatin in brain lysates from AD patients compared to age-matched control subjects (FIGS. 1F-H). The most distinct pattern of Aggregatin immunostaining observed in AD was that Aggregatin was remarkably immunoreactive within the center of amyloid plaques, which were stained by the pan-Aβ antibodies 6E10 and 4G8, the N-terminal truncated and modified pyroglutamate Aβ species Aβ [N3pe] antibody 82E1, fibrillar Aβ dye thioflavin-S (Thio-S) or oligomer Aβ antibody NU-4 (FIGS. 2A, B and FIGS. 3A-E). In contrast, all control brain sections lacking detectable amyloid plaques demonstrated weak diffusive Aggregatin immunoreactivity without association with puncta (FIG. 2A).

Robust Aggregatin staining of the central core of amyloid deposits was consistently observed in the brains of multiple mouse models for AD including 5×FAD, TgCRND8, APP/PS1, Tg2576, and 3×Tg transgenic mice overexpressing human mutant APP along with or without human mutant PS1 (FIGS. 2C, D and FIGS. 3F-H). With the exception of 5×FAD or Tg2576 mice in which Aggregatin-positive foci were connected with wispy fibrils, Aggregatin within amyloid deposits of other transgenic mice showed negligible projecting fibrillar structures, similar as in human plaques. Despite the general localization of Aggregatin large puncta to the core of amyloid deposits, they highly co-localized with Aβ in 5×FAD mice but not in AD patients or TgCRND8 mice, together indicating that the processes contributing to amyloid deposition may be different in human and different animal models. Notably, the formation of Aggregatin puncta occurred concurrently with amyloid deposition, but was not present in the pre-depositing young 5×FAD mice (FIG. 3I). The characteristic Aggregatin positive core staining was abolished by the pre-absorption of primary antibodies with human recombinant Aggregatin protein (rAggregatin) purified by combined 10 K dialysis and size exclusion chromatography, but not Aβ1-42 peptides (FIG. 4A), further validating the specificity of the anti-Aggregatin antibody. To confirm the presence of Aggregatin within amyloid deposits, we isolated amyloid cores purified by sucrose density gradient fractionation of 2% sodium dodecyl sulfate (SDS) homogenized AD or 5×FAD mouse brains. Dot blot and immunoblot studies of proteins under native and denatured forms respectively confirmed the existence of full-length Aggregatin without noticeable cleaved products in the SDS resistant insoluble core-enriched fractions positive for 6E10 (FIG. 2E-H).

Aggregatin Physically Interacts with Aβ

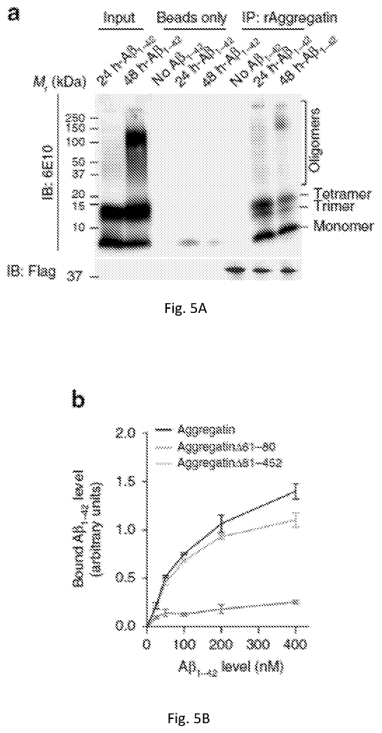

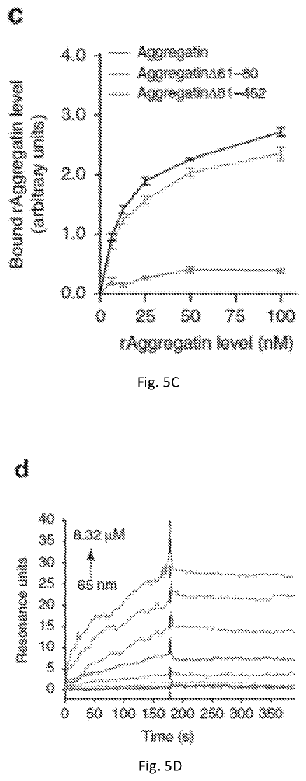

The radioimmunoprecipitation assay buffer (RIPA) widely used for coimmunoprecipitation failed to extract Aggregatin from AD brains (FIG. 4B), making it difficult to examine the likely association between Aggregatin and Aβ in AD. To overcome this obstacle, we performed in vitro pull-down assays using synthetic Aβ1-40 or Aβ1-42 and rAggregatin. Dynamic light scatting (DLS), circular dichroism (CD), and SDS-PAGE assays of rAggregatin indicated that rAggregatin existed in the soluble partially folded monomeric state (FIG. 4C-E). Notably, rAggregatin co-precipitated with different forms of Aβ1-40 or Aβ1-42 (FIG. 5A and FIG. 4F, G). Consistently, immobilized monomeric Aβ1-40 or Aβ1-42 was also able to pull down rAggregatin (FIG. 4H). Further surface binding affinity assays revealed that immobilized Aβ1-40 or Aβ1-42 bound to rAggregatin, and similarly, immobilized rAggregatin bound to Aβ1-40 or Aβ1-42 all within the nanomolar ranges (FIG. 5B, C and FIG. 4I, J). In agreement with these results, surface plasmon resonance (SPR) measurements confirmed that Aβ1-42 bound to immobilized rAggregatin at the low nanomolar dissociation equilibrium constant (Kd) (FIG. 5D). Although no measurement was noted in blank or BSA-immobilized sensor chips (FIG. 4K), signal spikes produced in the SPR assays may be in proportion to the mass of Aβ aggregates, making dynamic measurements unlikely consistent with the surface binding affinity assessments at the steady state. To investigate the binding of rAggregatin to Aβ ex vivo, we performed an in situ binding assay in which fixed brain sections of AD patients or 5×FAD mice were incubated with Flag-tagged rAggregatin and stained by an anti-Flag antibody. Remarkably, all amyloid deposits were labelled by rAggregatin (FIG. 4L-N). Considering the widespread presence of Aβ in brains, it was not surprising that brain sections also showed background staining after rAggregatin incubation. Notably, amyloid deposits and the background binding of rAggregatin were completely abolished by pre-incubation of rAggregatin with Aβ1-40 or Aβ1-42 (FIG. 4L-N), confirming that rAggregatin binds amyloid deposits by interacting with Aft Collectively, these results highlight the pathological relevance of Aggregatin in AD, and show that Aggregatin is an Aβ binding protein with high-affinity.

### Aggregatin Binds to Aβ Via its N-Terminal Region

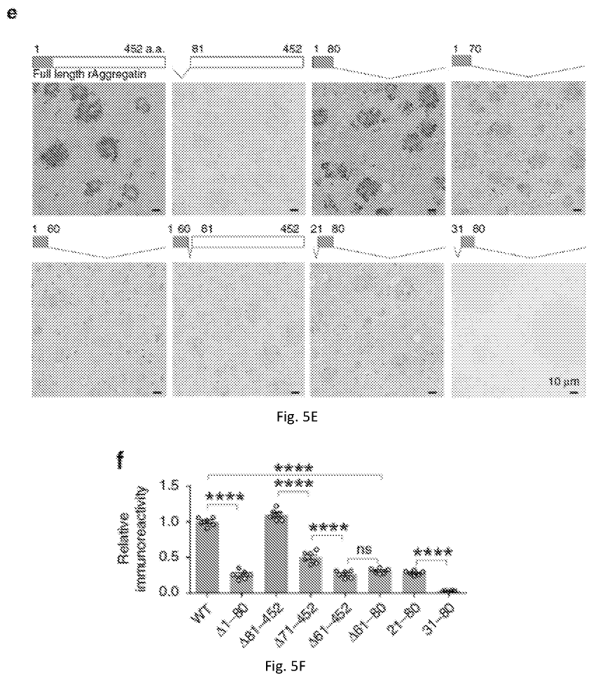

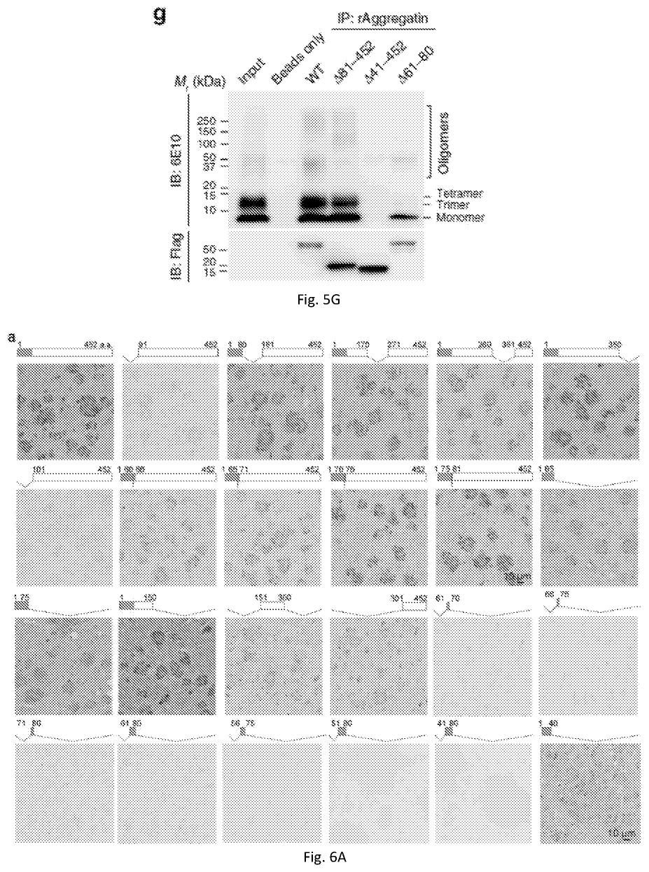

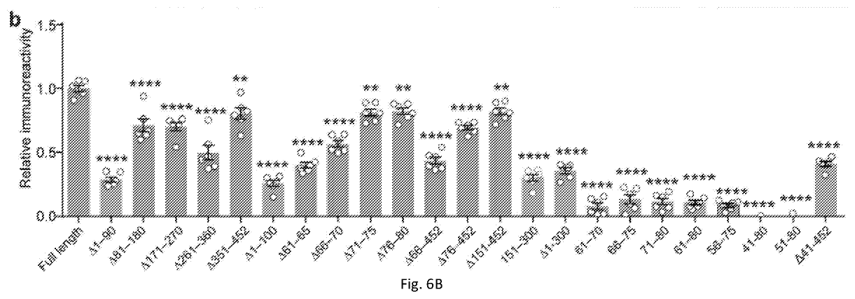

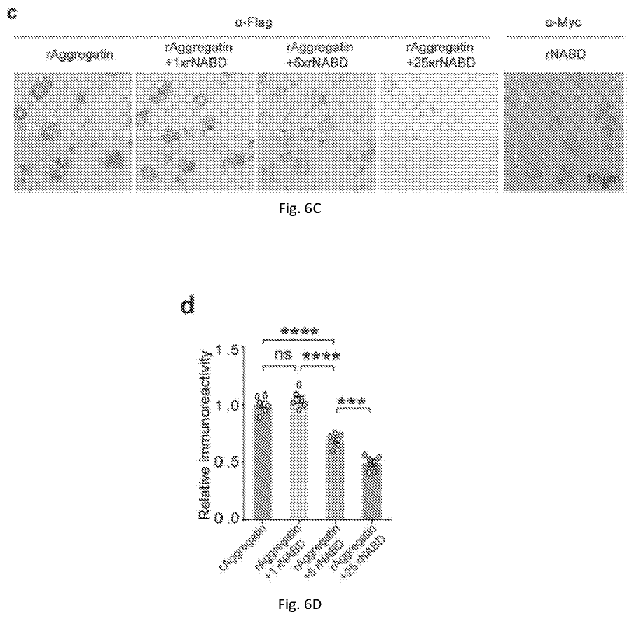

Next, we generated a series of rAggregatin deletion mutants to map the binding region for Aβ. Although rAggregatin alone does not form oligomers or aggregates, the composition of Aβ preparations at the micromolar range quickly changes over time due to the formation of higher order oligomers, which are expected to influence the Aggregatin and Aβ interaction. To quantitatively identify the binding strength of different rAggregatin deletion mutants, the in situ binding assay rather than pull-down assay was used for the binding motif mapping. The deletion of residues from 1 to 80 (designated as NABD, N-terminal Aβ binding domain), but not residues outside of this region, was found to greatly reduce the binding of rAggregatin to amyloid deposits (FIGS. 5E-G and FIGS. 6A, B). Recombinant NABD (rNABD) alone was able to bind to amyloid deposits or Aβ1-42 similar as full-length rAggregatin, and caused a dose-dependent decrease in the association between rAggregatin and amyl deposits when coincubated (FIG. 5B, C, E-G and FIG. 6C, D), together suggesting NABD as the domain both necessary and sufficient for Aβ binding. The residues from 61 to 80 appear to be a core motif for NABD though they alone were not sufficient to bind amyloid deposits (FIGS. 5E-G and FIGS. 6A, B). Notably, rNABD bound amyloid deposits in a length-dependent manner, and rAggregatin with partial deletions of every 5 amino acids within the core motif of NABD exhibited weaker but still strong interactions with amyloid deposits (FIGS. 6A, B), further indicating that NABD may contain multiple sites cooperatively involved in Aβ binding.

### Aggregatin Cross-Seeds Aβ Via Direct Binding

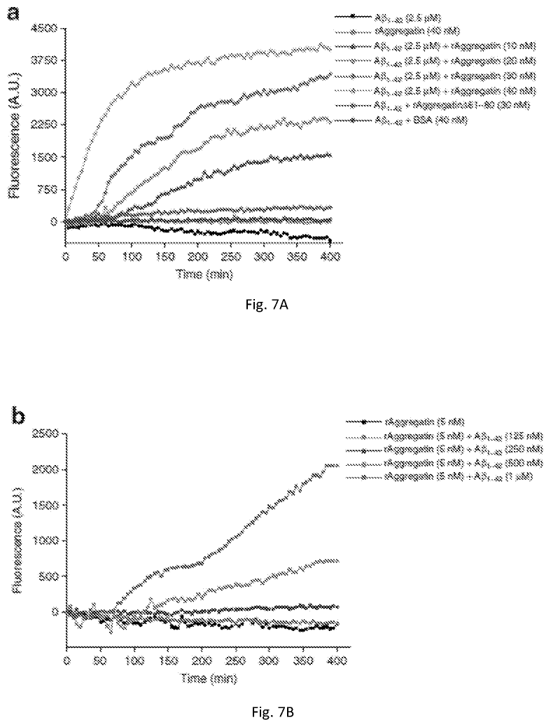

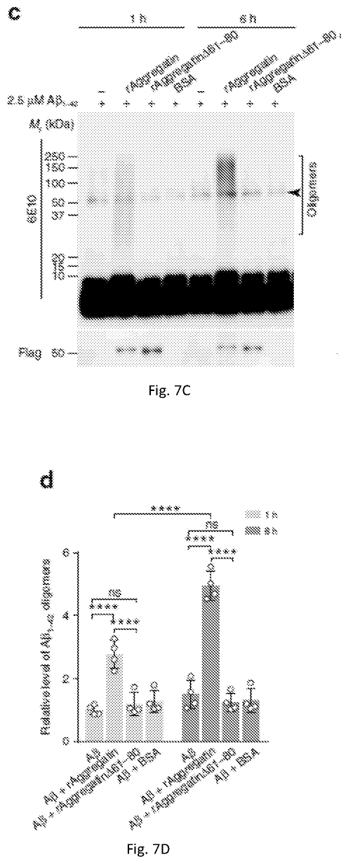

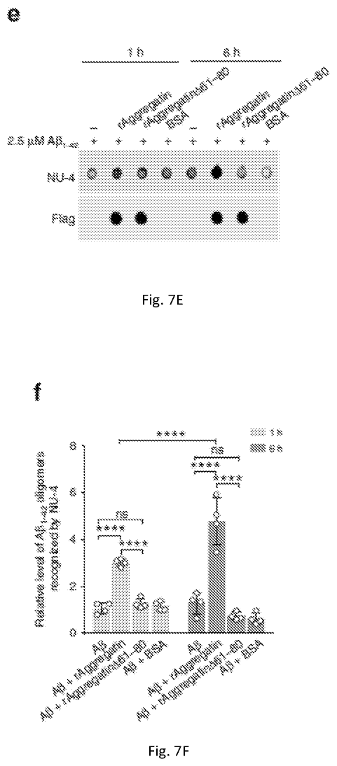

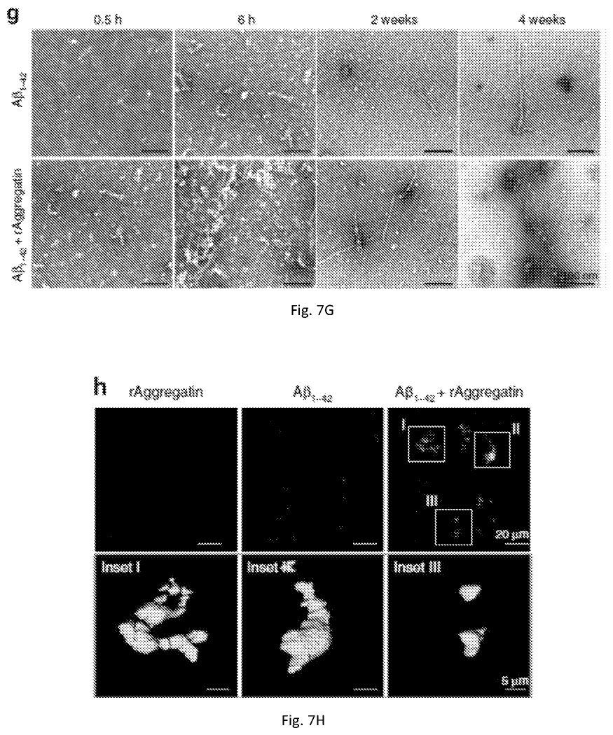

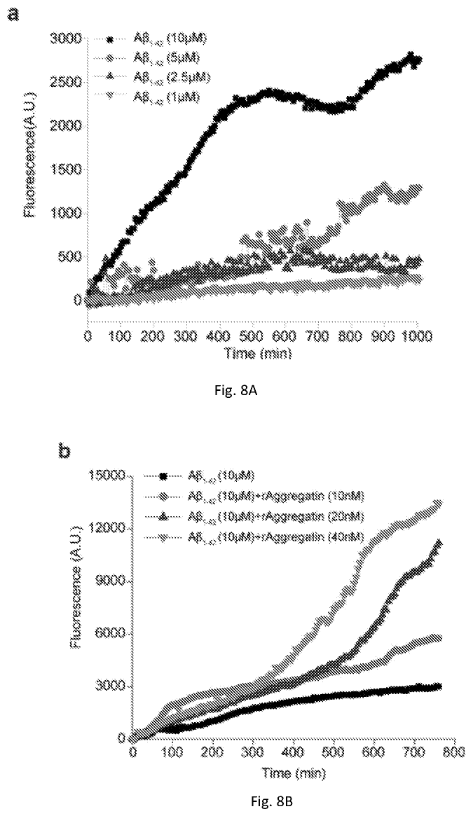

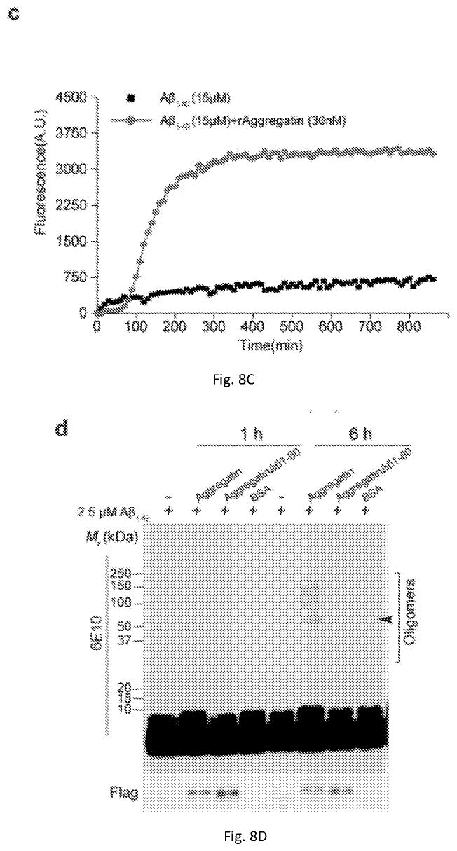

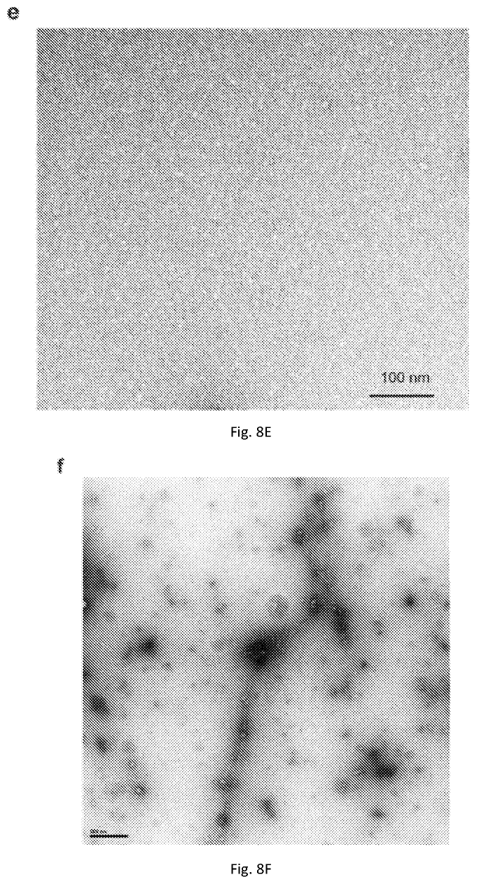

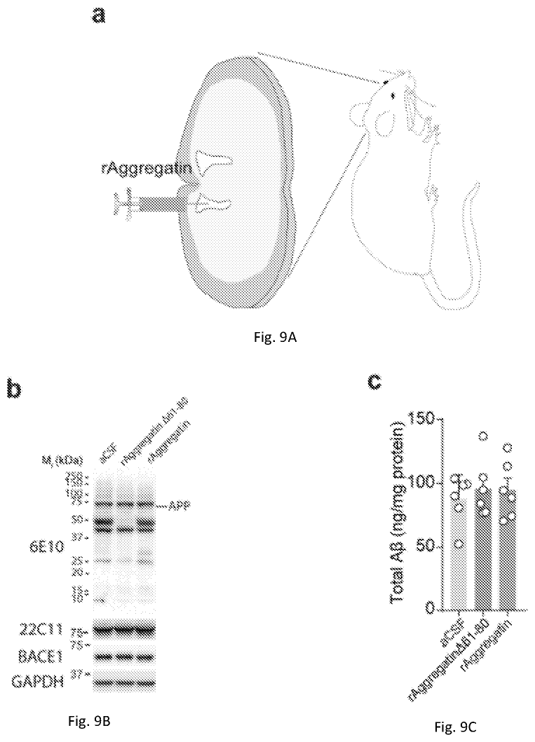

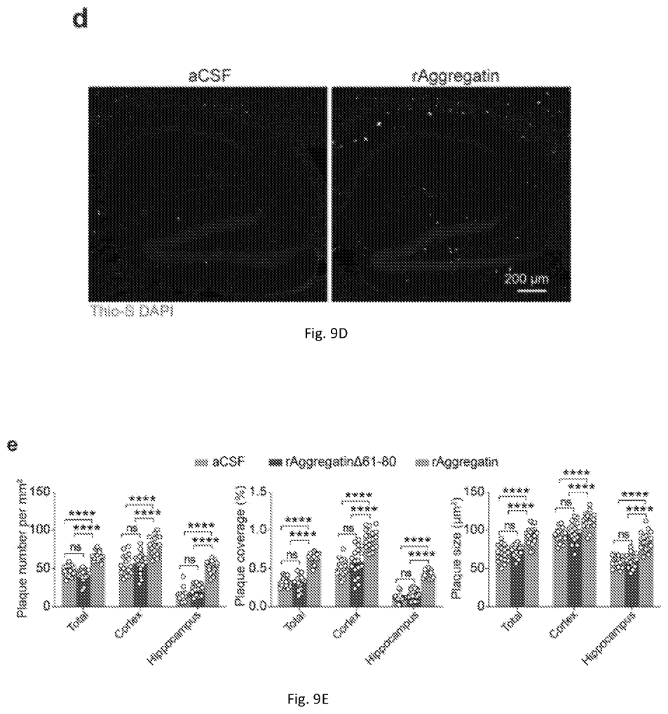

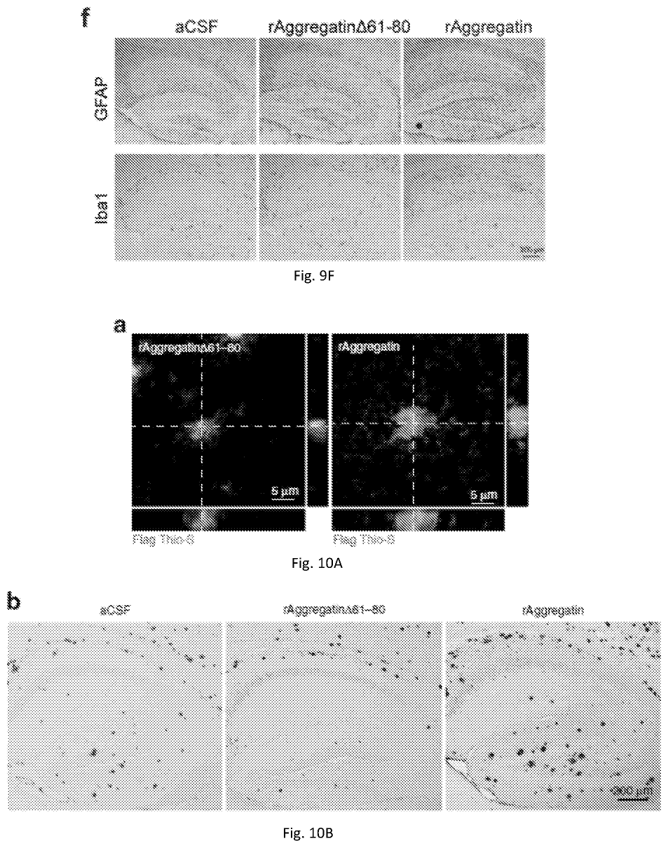

Given the strong interaction between Aggregatin and Aβ, we further set out to determine whether Aggregatin would influence the Aβ aggregation process. Aβ aggregation kinetics were first monitored in vitro using Aβ1-40 or Aβ1-42 for the thioflavin T (ThT) based fluorescence assay. As illustrated by changes in ThT-associated fluorescence, Aβ self-aggregated only at high concentrations whereas rAggregatin alone did not produce any observable aggregate (FIG. 7A and FIG. 8A). Remarkably, once co-incubated with rAggregatin, Aβ was able to form aggregates at low concentrations even in the nanomolar range (FIGS. 7A, B and FIG. 8A-C). With increasing concentrations of rAggregatin, the lag times of the aggregation reaction were greatly decreased (FIG. 7A and FIG. 8B). As a control, rAggregatinΔ61-80 had similar folding as wild type rAggregatin, but failed to induce Aβ1-42 aggregation (FIG. 7A and FIGS. 8D, E). These observations were confirmed using immunoblot and dot blot analyses for Aβ aggregation measurements under denatured and native conditions, which showed that Aggregatin but not rAggregatinΔ61-80 indeed promoted Aβ1-42 oligomerization (FIG. 7C-F and FIG. 8D). Of note, due to the sensitivity of immunoblot, Aβ oligomer was only detectable with long exposure when Aβ1-42 at the low micromolar but not nanomolar was applied. Consistently, transmission electron microscopy analyses revealed that soluble Aβ1-42 protofibrils were more abundant and have more complicated structures in the presence of rAggregatin during the early phase of incubation when Aβ fibrils were absent (FIG. 7G). As expected, the low concentration of Aβ1-42 only yielded very few short and un-branched fibrils after long periods of incubation under negative staining (FIG. 7G), and rAggregatin alone did not form identifiable particles or large aggregates (FIG. 9E). Strikingly, co-incubation of low micromolar Aβ1-42 with rAggregatin lead to the formation of large micrometer-long branched fibrils (FIG. 7G and FIG. 8F), which were Thio-Spositive and visible under the fluorescent microscopy (FIG. 7H). Taken together, these data imply Aggregatin as a potent seeding factor for Aβ oligomerization and aggregation.

### Aggregatin Regulates Amyloid Deposition

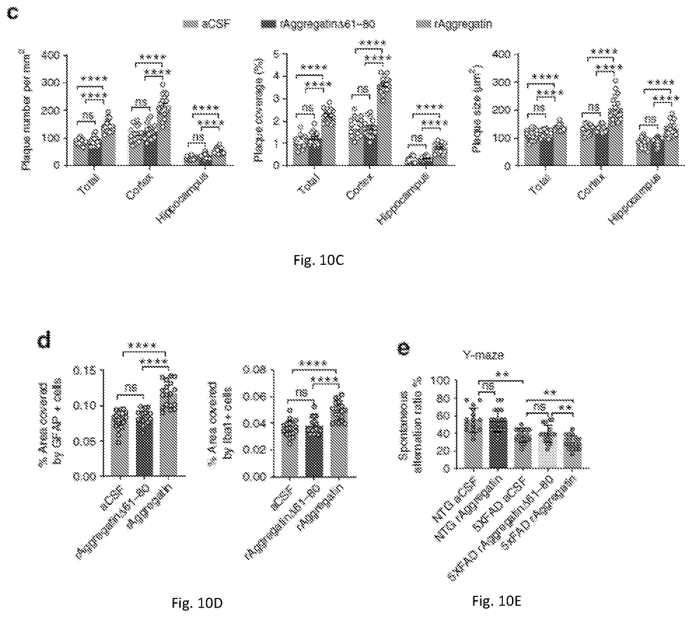

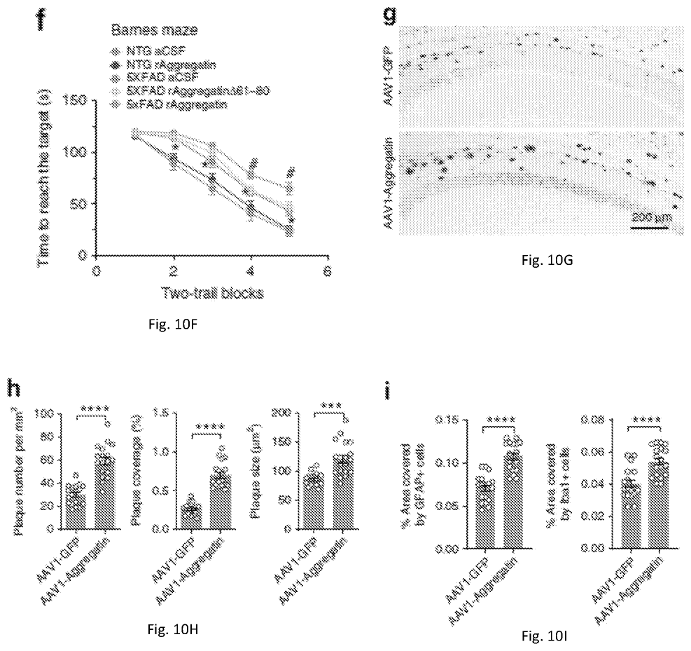

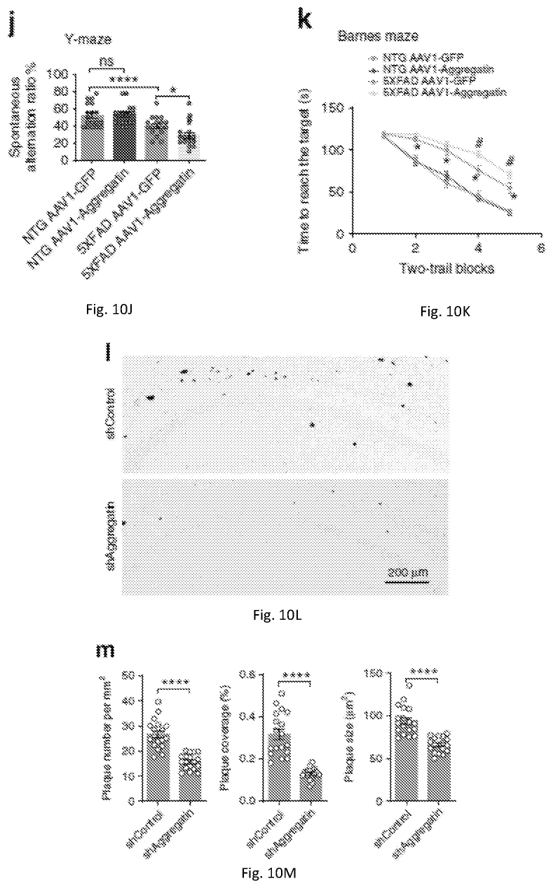

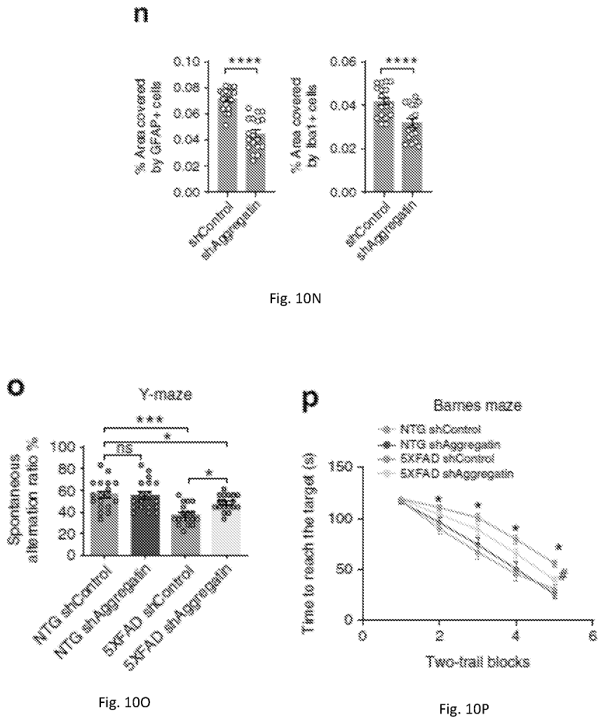

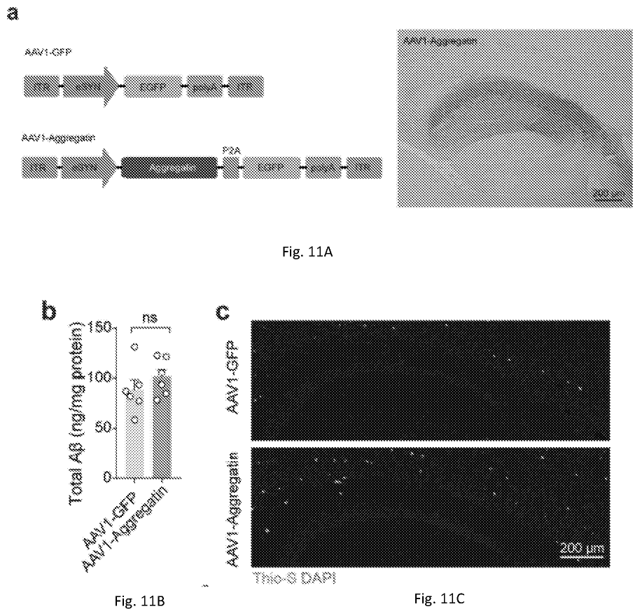

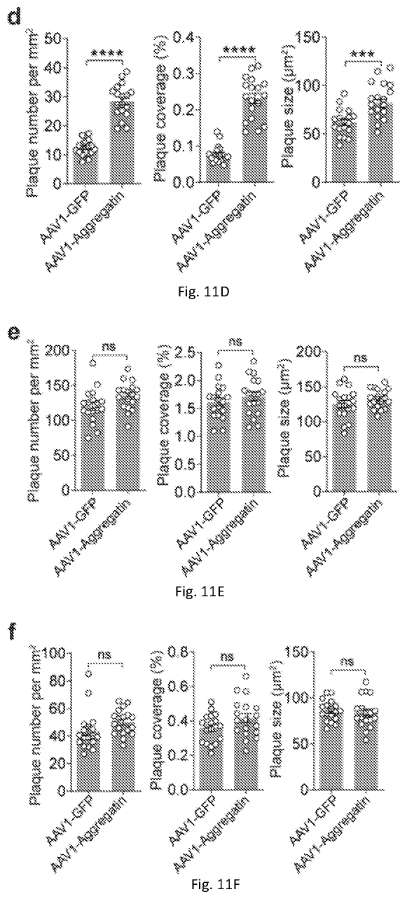

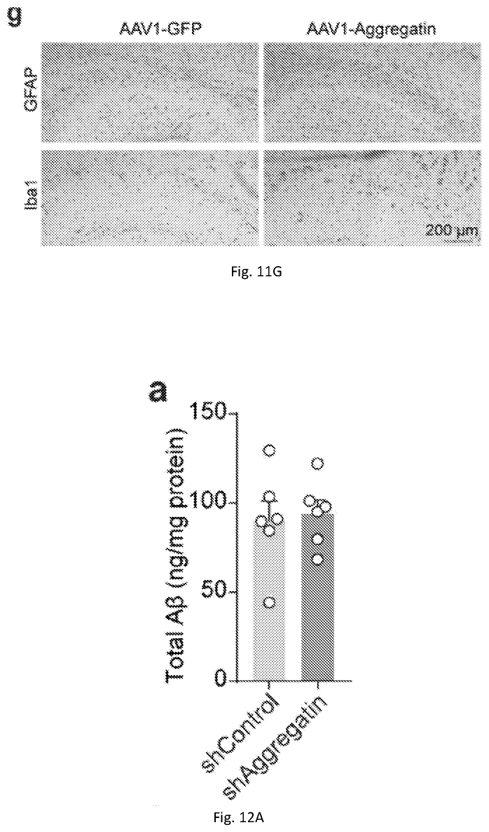

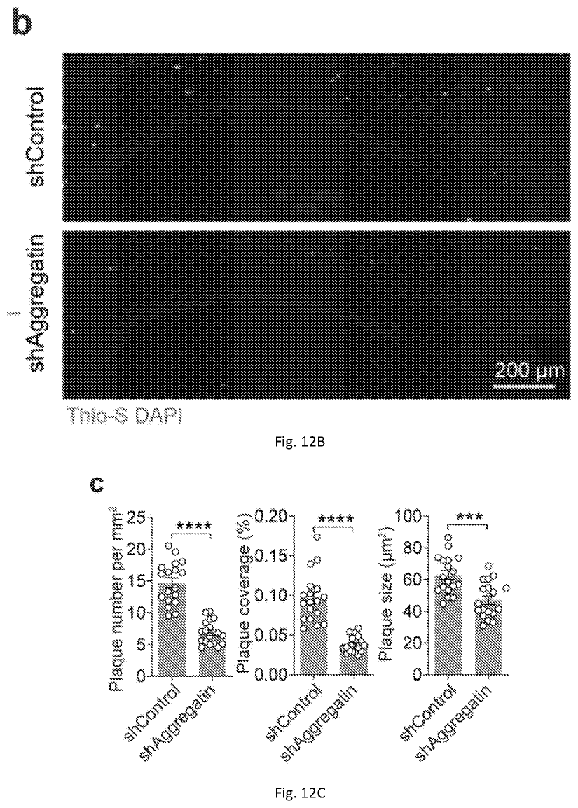

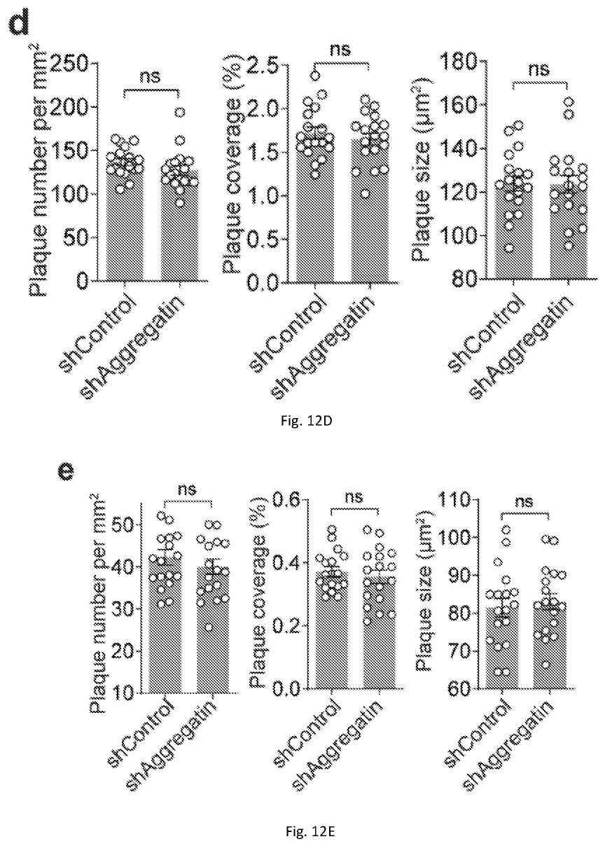

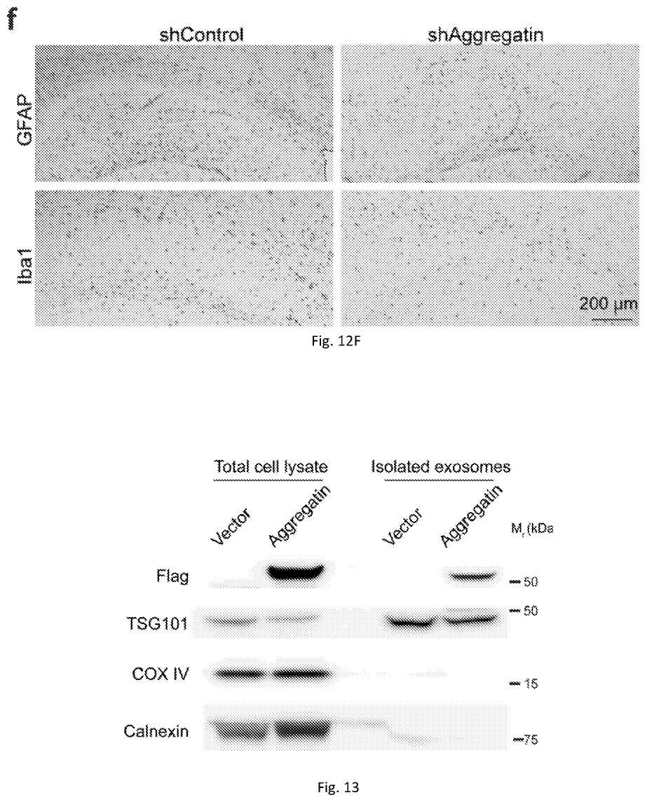

Aβ levels are low in young especially predepositing 5×FAD mice. To examine the effect of extracellular Aggregatin on amyloid deposition with unrestricted access to predeposit-state Aβ, we performed intracerebroventricular (ICV) infusion of Flag-tagged rAggregatin or rAggregatinΔ61-80 into 5×FAD mice at 4-month-old, when Aβ rises to high levels (FIG. 9A). Infusion did not cause the death of mice or histological abnormalities in the brain. Importantly, the levels of total Aβ, APP or BACE1 remained unchanged 4 weeks after rAggregatin infusion, indicating that rAggregatin did not affect Aβ production or degradation (FIG. 9B, C). ICV infused rAggregatin was detected in amyloid deposit (FIG. 10A). Remarkably, compared to age-matched control mice infused with artificial cerebrospinal fluid (aCSF), rAggregatin-infused mice showed greatly increased amyloid deposition spreading the brain at 5 months of age, which could be completely blocked by the deletion of NABD core motif (FIGS. 10B, C and FIGS. 9D, E). As prominent AD pathological features, microgliosis and astrogliosis are closely associated with amyloid deposits in 5×FAD mice. Corresponding to increased plaque load, 5×FAD mice infused with rAggregatin but not rAggregatinΔ61-80 exhibited more microgliosis and astrogliosis compared to aCSF-infused control 5×FAD mice (FIG. 10D and FIG. 9F). 5×FAD mice begin to show cognitive deficits at around 4-months-old. Compared with NTG mice, FAD mice exhibited significantly impaired Y-maze and Barnesmaze performance, both of which were significantly exacerbated in transgenic mice with rAggregatin but not rAggregatinΔ61-80 infusion (FIG. 10E, F). To further examine the role of neuronal Aggregatin in amyloid deposition, we injected adeno-associated virus serotype 1 encoding human Aggregatin or GFP alone under the neuron specific promoter eSYN (AAV1-Aggregatin or AAV1-GFP) into the hippocampus CA1 of young predepositing 5×FAD mice at 1.5-month-old (FIG. 11A). When analyzed at 5 months of age, in line with ICV infusion experiments, intrahippocampal injection of AAV1-Aggregatin significantly increased amyloid deposition without any effect on total Aβ levels in the GFPpositive hippocampal region, but not in the brain areas without AAV1-Aggregatin delivery (FIGS. 10G, H and FIGS. 11B-F), together suggesting that Aggregatin is sufficient to enhance amyloid deposition in vivo. Consistently, amyloid deposition associated microgliosis, astrogliosis, and cognitive deficits were also worsened by neuronal Aggregatin overexpression (FIGS. 10I-K and FIG. 11G). To investigate whether Aggregatin was required for amyloid deposition, we performed intrahippocampal injection of AAV1 co-expressing GFP and a short hairpin RNA targeting Aggregatin (AAV1-shAggregatin) or control shRNAi (AAV1-shControl) in predepositing 5×FAD mice (FIGS. 10B, C). It was observed that decreasing Aggregatin was not associated with neuronal loss or altered total Aβ (FIG. 12A). At 5 months of age, the injection of AAV1-shAggregatin significantly alleviated amyloid deposition in the GFP-positive areas of hippocampus compared to AAV1-shControl injection, but not in the GFP-negative brain areas (FIG. 10L, M and FIGS. 12B-E). Likewise, Aggregatin reduction significantly alleviated amyloid deposit associated microgliosis, astrogliosis, and cognitive impairment (FIG. 10N-P and FIG. 12F). Taken together, these results further imply that Aggregatin is also an important factor necessary for amyloid deposition.

We show Aggregatin, the protein encoded by FAM222A, as a plaque core protein directly binding Aβ and facilitating Aβ aggregation, a process thought to be central in AD onset. Therefore, this work provides strong experimental evidence supporting a pathophysiological role for Aggregatin in AD.

In people diagnosed with AD or mild cognitive impairment (MCI), a proportion of whom can progress to AD, FAM222A is associated with the module enriched for atrophy in AD-affected brain regions. FAM222A association with hippocampal volume could be validated in the replication ENIGMA cohort, together pointing to a potential mechanism by which FAM222A may affect regional brain atrophy. Notably, our cross phenotype association analysis also led to the identification of long-established AD risk genes APOE, TOMM40, and APOC1 exclusively in the same module, suggesting possible genetic interplays between FAM222A and AD risking genes. Interestingly, although we only discovered marginal association between rs117028417 and AD diagnosis, FAM222A, but not the nearby gene TRPB4, was found significantly associated with longitudinal increase of brain amyloid deposition. Along this line, as AD is a genetically complex and multifactorial disease with different etiological subtypes, FAM222A variants or pathogenic mutations strongly associated with AD may be present in subsets of AD patients. Nevertheless, although our genetic discovery study did not observe a strong influence of FAM222 variant on AD risk and biomarkers, the module enriched for FAM222A and previously reported AD risk variants likely represents a statistical AD-specific cluster worthy of further investigation using independent AD neuroimaging databases.

Consistent with the genetic association of FAM222A with longitudinal brain Aβ deposition, pathologically accumulated Aggregatin, the protein encoded by FAM222A, is readily noted in plaques in AD and amyloid deposits in multiple APP transgenic mice, strongly illustrating the pathological function of Aggregatin. Of note, there are remarkable differences in the morphology of Aggregatin puncta and their co-localization with Aβ. Similarly, as plaques in AD patients are more complex structures than amyloid deposits in APP transgenic mice, it could be expected that Aggregatin is also present differentially in amyloid core-enriched fractions from AD patients and 5×FAD mice. A number of explanations may account for the discrepancy regarding the pattern of Aggregatin puncta or presence of Aggregatin in plaques, including but not limited to differences in disease stages, the effects of Aβ clearance and degradation pathways or the length of time spent for plaque deposition. This notion is indeed supported by the observation that while only one or several condensed Aggregatin foci were present in single plaque in AD, amyloid deposits in cortex from patients with Down's syndrome (DS), a complex genetic abnormality developing AD-like pathology, were largely associated with multiple foci.

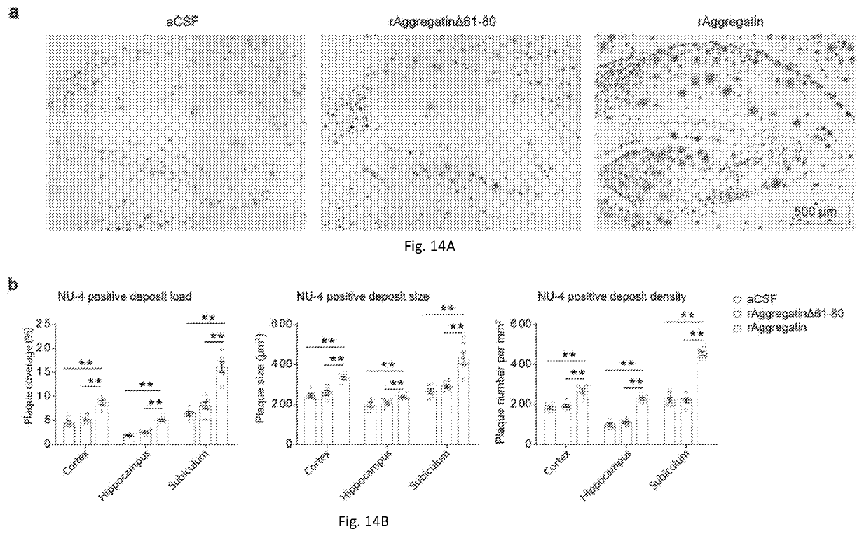

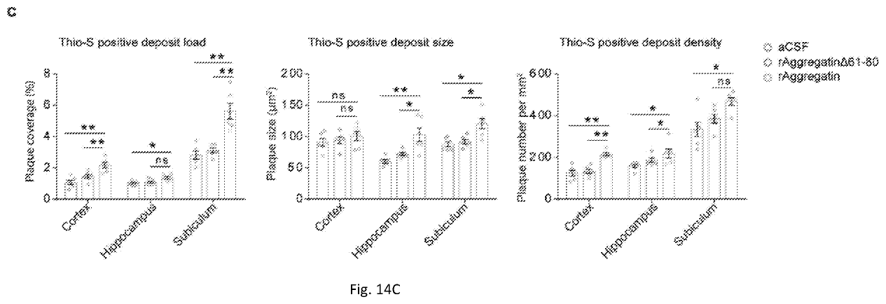

It is still unclear how Aggregatin becomes accumulated within the center of plaques without the ability for self-aggregation. Aggregatin appears to bind Aβ1-40 and Aβ1-42 with different affinities. Along this line, amyloid plaques are made up of different N or C-terminally truncated and modified Aβ species. Interestingly, we found that Aggregatin was present in exosomes (FIG. 14). Although Aggregatin has no signal sequence and is not predicted to be secreted, this data supports the possibility that Aggregatin can be exported into the interstitial fluid. Of note, the presence of exogenously expressed Aggregatin in exosomes of cultured cells is physiologic. There may be other mechanisms responsible for Aggregation secretion under pathological conditions. As Aggregatin protein levels were upregulated in AD, there may be a complex interplay among Aβ specific forms, Aggregatin expression, post-translational modification, extracellular secretion, and other unknown factors responsible for this. Nevertheless, on the basis of the facts that Aggregatin puncta appear concurrently with amyloid plaques and does not exist in the predepositing mice, Aggregatin should accumulate in plaques before or concurrent with rather than after the well formation of plaques. Aggregatin did not form intraneuronal accumulation in AD patients and 5×FAD mice. Not surprisingly, we did not observe the presence of Aggregatin puncta in neurons bearing neurofibrillary tangles. Along this line, intraneuronal APP and/or Aβ immunoreactivity assessed by 6E10 was not changed by Aggregatin in 5×FAD mice. Therefore, Aggregatin may not be involved in intraneuronal protein aggregation. Noteworthily, Aggregatin does not physically interact with tau and other previously reported plaque-associated proteins such as α-synuclein and APOE, further implicating the likely specific link between Aggregatin and Aβ. However, as AD is a multifactorial disease, further detailed investigation will still be needed to determine the spatiotemporal relationship between Aggregatin and other AD-related pathologies especially considering the presence of Aggregatin immunoreactivity outside of plaques.

Aggregatin facilitates Aβ aggregation in vitro although it is not clear whether Aggregatin influences the primary or secondary nucleation. Increasing Aggregatin enhances, whereas reduced Aggregatin suppresses amyloid deposition and associated neuroinflammation and cognitive deficits. Of note, in addition to exacerbate Aβ pathology in adult 5×FAD mice, Aggregatin infusion causes further amyloid deposition in aged 5×FAD mice when amyloid deposit size and number largely plateau (FIG. 14). Therefore, Aggregatin is likely an unrecognized co- or even limiting factor both necessary and sufficient for Aβ aggregating into the fibrils to form plaques. Although the bioinformatics analysis of Aggregatin amino acid sequence reveals that Aggregatin does not contain any known conserved functional motifs, our CD characterization of Aggregatin indicated it as at least a partially folded protein containing α-helix, β-sheet, and intrinsically disordered element(s). While the structure and physiological function of Aggregatin is still under investigation, we found that Aggregatin was exclusively expressed in the CNS. The substantial loss of Aggregatin in hippocampus does not cause neuronal death, suggesting that Aggregatin may not be vital for neuronal survival.

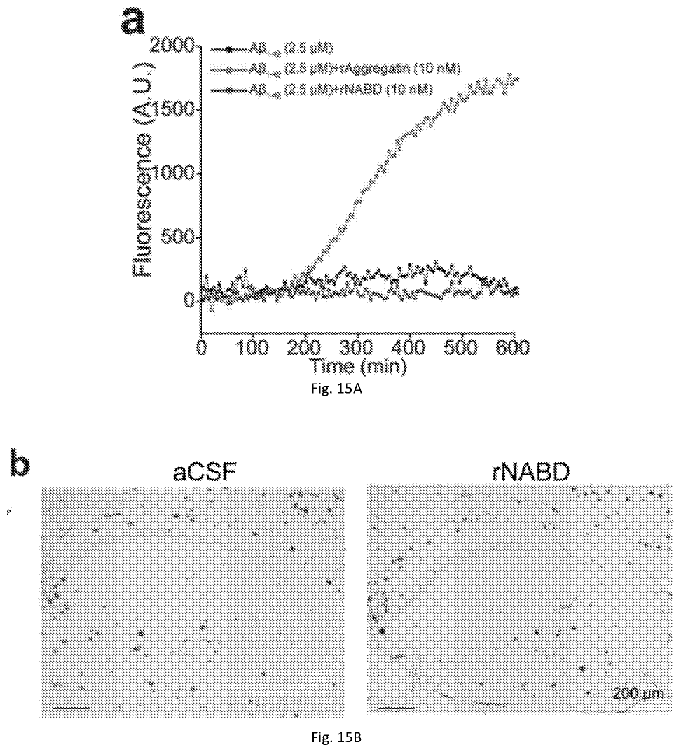

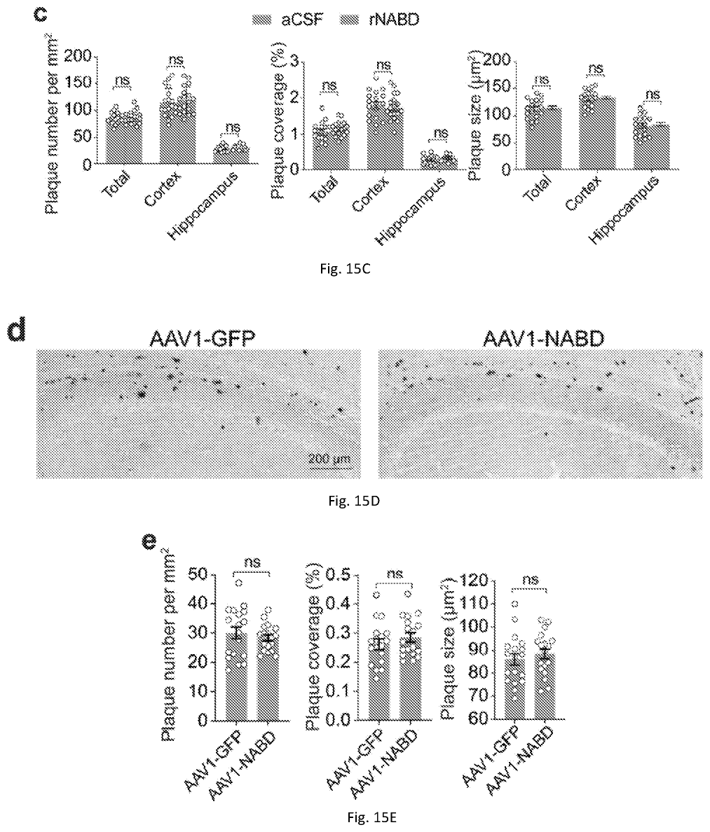

The genetic inhibition of Aggregatin-Aβ interaction was able to suppress Aggregatin-induced Aβ aggregation or amyloid deposits, suggesting that Aggregatin should directly interact with Aβ to regulate its pathology. Of note, although rNABD (i.e. Aggregatin 1-80 or Aggregatin 481-452) alone is able to bind Aβ, it does not induce Aβ1-42 aggregation or promote amyloid deposits (FIG. 15), suggesting that the C-terminal fragment is also required for Aggregatin-induced Aβ aggregation and plaque formation. The exact mechanism for Aggregatin-mediated Aβ aggregation is still under investigation.

In conclusion, we have shown FAM222A as a gene associated with AD-related regional brain atrophy, which encodes an amyloid plaque core protein pathologically involved in Aβ assembly and amyloid deposition.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of identifying a subject at risk of a disease or disorder associated with amyloid aggregation, the method comprising:
assaying for Aggregatin in a bodily sample obtained from the subject, wherein the subject is at risk of having the disease or disorder if the Aggregatin is present above a threshold level.

2. The method of claim 1, wherein the subject is not at risk of having the disease or disorder if the Aggregatin is not above a threshold level.

3. The method of claim 1, wherein the disease or disorder is associated with amyloid β aggregation.

4. The method of claim 3, wherein the disease or disorder is neurodegenerative disease or disorder.

5. The method of claim 1, wherein the disease or disorder comprises at least one of Alzheimer's disease (AD), Alzheimer's related dementia, Parkinson's disease, Huntington's disease, amyotrophic lateral sclerosis (ALS), Lewy body dementia (LBD), or Down's syndrome.

6. The method of claim 1, wherein the disease or disorder is Alzheimer's disease.

7. The method of claim 1, wherein the bodily sample comprises blood, serum, plasma, urine, cerebrospinal fluid (CSF), synovial fluid, or spinal fluid.

8. The method of claim 1, wherein the bodily sample blood, serum, or plasma.

9. The method of claim 1, wherein the bodily sample is treated with a protease to obtain peptide fragments of Aggregatin and the presence or level the peptide fragments is detected by mass-spectrometry to determine the presence or level of Aggregatin in the bodily sample.

10. The method of claim 9, wherein the peptide fragments are chromatographically separated from other components in the sample by liquid chromatography.

11. The method of claim 9, wherein the peptide fragments include peptides having the amino acid sequences of SEQ ID NO: 3 and SEQ ID NO: 4.

12. The method of claim 11, wherein the ratio of the peptide fragments having SEQ ID NO: 3 and SEQ ID NO: 4 is determined by mass spectrometry and the determined ratio is compared with a standard curve generated from mass spectrometric results for known ratios of synthetic peptides having SEQ ID NO: 3 and SEQ ID NO: 4 to determine the presence or level of Aggregatin in the sample.

13. The method of claim 1, wherein the bodily sample is blood, serum, or plasma and the presence of the Aggregatin in the bodily is indicative of the subject being at risk of the disease or disorder.

14. A method of detecting a disease or disorder associated with amyloid aggregation, the method comprising:
assaying for Aggregatin in a bodily sample obtained from the subject, wherein the subject is having the disease or disorder if the Aggregatin is present above a threshold level.

15. The method of claim 14, wherein the subject does not have the disease or disorder if the Aggregatin is not above a threshold level.

16. The method of claim 14, wherein the disease or disorder is associated with amyloid β aggregation.

17. The method of claim 16, wherein the disease or disorder is neurodegenerative disease or disorder.

18. The method of claim 14, wherein the disease or disorder comprises at least one of Alzheimer's disease (AD), Parkinson's disease, Huntington's disease, amyotrophic lateral sclerosis (ALS), Lewy body dementia (LBD), or Down's syndrome.

19. The method of claim 14, wherein the disease or disorder is Alzheimer's disease.

20. The method of claim 14, wherein the bodily sample comprises blood, serum, plasma, urine, cerebrospinal fluid (CSF), synovial fluid, or spinal fluid.

21. The method of claim 14, wherein the bodily sample blood, serum, or plasma.

22. The method of claim 14, wherein the bodily sample is treated with a protease to obtain peptide fragments of Aggregatin and the presence or level the peptide fragments is detected by mass-spectrometry to determine the presence or level of Aggregatin in the bodily sample.

23. The method of claim 22, wherein the peptide fragments are chromatographically separated from other components in the sample by liquid chromatography.

24. The method of claim 22, wherein the peptide fragments include peptides having the amino acid sequences of SEQ ID NO: 3 and SEQ ID NO: 4.

25. The method of claim 24, wherein the ratio of the peptide fragments having SEQ ID NO: 3 and SEQ ID NO: 4 is determined by mass spectrometry and the determined ratio is compared with a standard curve generated from mass spectrometric results for known ratios of synthetic peptides having SEQ ID NO: 3 and SEQ ID NO: 4 to determine the presence or level of aggregatin in the sample.

26. The method of claim 14, wherein the bodily sample is blood, serum, or plasma and the presence of the aggregatin in the bodily is indicative of the subject having the disease or disorder.

27-38. (canceled)
